(19) United States
(12) Patent Application Publication
(10) Pub. No.: US 2013/0344519 A1
(43) Pub. Date: Dec. 26, 2013

(54) METHODS AND SYSTEMS FOR ASSESSMENT OF TURBIDITY KINETICS (WAVEFORM ANALYSIS) IN COAGULATION TESTING

(71) Applicant: Bayer HealthCare LLC, Berkeley, CA (US)

(72) Inventors: Lilley Leong, Hercules, CA (US); Philip Ramsey, San Rafael, CA (US)

(21) Appl. No.: 13/926,966

(22) Filed: Jun. 25, 2013

Related U.S. Application Data
(60) Provisional application No. 61/663,912, filed on Jun. 25, 2012.
(60) Provisional application No. 61/793,864, filed on Mar. 15, 2013.

(51) Int. Cl. G01N 33/86

(52) U.S. Cl. CPC G01N 33/86
USPC 435/13; 435/288.7

(57) ABSTRACT

In some embodiments, a method is provided that includes (1) obtaining a plasma sample from a patient; (2) performing a coagulation assay on the plasma sample; (3) measuring a coagulation property of the plasma sample using a coagulation analyzer so as to generate measured data; (3) performing waveform analysis on the measured data so as to obtain turbidity characteristics; and (4) employing the waveform analysis to determine a coagulation status of the coagulation assay not provided by the coagulation analyzer. Numerous other embodiments are provided.

The present application claims priority to U.S. Provisional Patent Application Ser. No. 61/663,912, filed Jun. 25, 2012 and titled “METHODS AND APPARATUS FOR USING WAVEFORM ANALYSIS FOR COAGULATION TESTING” and U.S. Provisional Patent Application Ser. No. 61/793,864, filed Mar. 15, 2013 and titled “METHODS AND SYSTEMS FOR ASSESSMENT OF TURBIDITY KINETICS (WAVEFORM ANALYSIS) IN COAGULATION TESTING,” each of which is hereby incorporated by reference herein in its entirety for all purposes.

### BACKGROUND

Blood coagulation occurs in response to vascular injury and involves the in-situ generation of a plasma enzyme thrombin. The thrombin generated cleaves its plasma protein substrate fibrinogen to yield fibrin. The fibrin polymer generated has characteristic physical properties, including turbidity, depending on fiber size and organization. Development of physical polymer properties, therefore, not only tracks the progress of coagulation, but also provides an indirect gauge on fibrin polymer structure.

Blood coagulation tests rely on measurement of thrombin generated or on development of fibrin turbidity or viscoelasticity. Tests relying on the latter principles form the basis of clinical blood coagulation tests for a variety of applications, including, for example, when patients undergo surgery, or when coagulation abnormality (such as bleeding or hypercoagulation) is suspected in a patient or when patient response to procoagulant or anticoagulant therapy is monitored. In addition, coagulation testing is also performed in the context of drug discovery for new procoagulants and anticoagulants.

When used alone, conventional coagulation assays, such as activated partial thromboplastin time (“aPTT”) assay, prothrombin time (“PT”) assay, and variations of these two assays, may not be sufficiently reliable and/or may provide insufficient information on fibrin polymer characteristics for some applications. Coagulation analyzers for use in such conventional assays typically use mechanical or optical methods to detect development of viscoelasticity (mechanical detection) or turbidity (optical detection) in response to an activator of coagulation in the coagulation tests. Clot times (“CT”) are recorded when the patient sample achieves a predefined turbidity or viscosity when fibrin polymerizes into a gel in an optical or mechanical analyzer. However, clot times CT alone may not provide sufficient insights into coagulation processes and mechanisms.

As such, improved systems and methods for coagulation testing are needed.

### SUMMARY

In some embodiments, a method includes (1) obtaining a plasma sample from a patient; (2) performing a coagulation assay on the plasma sample; (3) measuring a coagulation property of the plasma sample using a coagulation analyzer so as to generate measured data; (4) performing waveform analysis on the measured data so as to obtain turbidity characteristics; and (5) employing the waveform analysis to determine a coagulation status of the coagulation assay not provided by the coagulation analyzer. Numerous other embodiments are provided.

These and other features of the present teachings are set forth herein.

### DRAWINGS

The skilled artisan will understand that the drawings, described below, are for illustration purposes only. The drawings are not intended to limit the scope of the present teachings in any way.

FIG. 1 is a schematic diagram of an example system for performing waveform analysis on coagulation data in accordance with various embodiments.

FIG. 2 is a flowchart illustrating a first example method of performing waveform analysis on coagulation data in accordance with various embodiments.

FIG. 3 is a flowchart illustrating a second example method of performing waveform analysis on coagulation data in accordance with various embodiments.

FIG. 4 is flow diagram illustrating a third example method for conducting waveform analysis using coagulation data in accordance with various embodiments.

FIG. 5A illustrates normalized data for a plasma sample, showing optical density (“OD”) change vs. time in accordance with various embodiments.

FIG. 5B illustrates the first derivative transformation of the measured data of FIG. 5A (top graph of FIG. 5B) and the second derivative transformation of the measured data of FIG. 5A (bottom graph of FIG. 5B) in accordance with various embodiments. The first derivative is the source of the CT for the PT assay, while the second derivative provides the CT for the aPTT assay, with the coagulation analyzer used in this example.

FIGS. 6A-B show graphically the heterogeneity of individual HemA plasma in accordance with various embodiments.

FIG. 7A shows graphically waveform analysis of example plasma samples that failed to yield aPTT CT results. The rate of turbidity development (first derivative transform) is depicted for these samples in accordance with various embodiments.

FIG. 7B shows graphically CT determination of the plasma described in FIG. 7A. The second derivative (acceleration of turbidity development) used for CT determination is depicted here in accordance with various embodiments.

FIG. 8 shows graphically HemA plasma spiked with FVIII have different turbidity maxima in accordance with various embodiments.

FIG. 9A shows graphically correlation of turbidity maxima with clot times in some HemA plasma containing 25% FVIII (modeled by spiking in rFVIII protein into FVIII-deficient HemA plasma) in accordance with various embodiments. As 25% FVIII is thought to be sufficient to confer near-normal coagulation, the turbidity maxima versus CT of normal plasma (containing 100% FVIII) was included as a point of reference.

FIG. 9B shows graphically turbidity maxima and plasma fibrinogen levels are directly correlated in individual HemA plasma described in FIG. 9A in accordance with various embodiments.

FIG. 9C shows graphically a scatter plot of CT vs. maximum acceleration of turbidity in the plasma described in FIG. 9A. Again normal plasma is also included as a point of comparison. This figure demonstrates that compared to normal plasma, HemA plasma has markedly reduced acceleration of turbidity development, in accordance with various embodiments.

FIG. 10A shows graphically correlation of turbidity maxima with clot times in some HemA plasma containing 1% FVIII in accordance with various embodiments. Plasma FVIII at the 1% level is the level below which the frequency of spontaneous bleeding occurs.

FIG. 10B shows correlation of maxima rate of turbidity development and clot times in the HemA plasma described in FIG. 10A in accordance with various embodiments.

FIG. 10C shows graphically a scatter plot of CT vs. maximum acceleration of turbidity in the plasma described in FIG. 10A, demonstrating the marked reduction (˜100 AU vs. 600 AU of normal in FIG. 10C) in acceleration with the 1% FVIII level in accordance with various embodiments.

FIGS. 10D-E show the thrombin response of plasma containing≦1% FVIII from the indicated hemophilia A donors in accordance with various embodiments.

FIGS. 11A-B show graphically waveform analysis of aPTT can discriminate between rFVIII, rFVIIa, or BAY 86-6150 in plasma from HemA donors with 112 BU inhibitors (anti-FVIII antibodies) using absorbance data (FIG. 11A) and the first derivative transformation of absorbance data (FIG. 11B) in accordance with various embodiments.

FIGS. 12A and 12B demonstrate the application of waveform analysis to dissect out potential mechanisms of action for coagulation proteins in HemA patient with inhibitors. FIG. 12A shows graphically the waveform analysis of the PT assay to assess coagulation with rFVIII, rFVIIa, or BAY 86-6150 together in accordance with various embodiments. The PT assesses the extrinsic portion of coagulation. FIG. 12B shows graphically discrimination of differential mode of action (more intrinsic pathway involvement) for new BAY 86-6150 in development, using aPTT and waveform analysis together in accordance with various embodiments.

FIGS. 13A-13D illustrate the application of waveform analysis to detect anticoagulant reversal with a commercially available procoagulant, ProfilIX. FIG. 13A shows maximum turbidity versus an FXa-specific inhibitor BAY794983 dose in accordance with various embodiments. FIG. 13B shows maximum rate of turbidity development versus BAY794983 dose in accordance with various embodiments. FIG. 13C shows difference in maximum turbidity with and without ProfilIX versus BAY794983 dose in accordance with various embodiments. The inset shows the difference in CT with and without ProfilIX as a function of BAY794983. FIG. 13D shows difference in maximum rate of turbidity development with and without ProfilIX versus BAY794983 dose in accordance with various embodiments.

FIG. 14 shows graphically that FVIII, FVII, von Willebrand disease (type 3) deficiency are detectable by waveform analysis on measured data from factor-specific assays, using first derivative transformation data in accordance with various embodiments.

### DESCRIPTION OF VARIOUS EMBODIMENTS

As stated, coagulation analyzers typically generate clot times for a given coagulation assay. This data alone may provide insufficient information regarding the coagulation processes and/or mechanisms being studied. However, additional information such as optical density or turbidity over time also can be generated with a coagulation analyzer for a given coagulation assay. This additional information is often unused. Waveform analysis subjects this unused optical or turbidity information to further analysis to characterize the coagulation process in the sample tested.

In accordance with embodiments described herein, optical density, turbidity and/or other data measured for a coagulation assay by a coagulation analyzer is collected, manipulated, transformed and/or otherwise analyzed to obtain additional insights into coagulation processes and/or coagulation mechanisms. Such collection, manipulation, transformation and/or other analysis processes performed on data measured by a coagulation analyzer are referred to collectively herein as “waveform analysis.”

Through use of waveform analysis, coagulation analyzer data that would otherwise be used primarily for clot time measurements can be used to gain insights into coagulation diagnostics, clotting dynamics, coagulation monitoring, drug discovery, and the like (generally referred to as “coagulation status”). Specific examples of waveform analysis applications for determining coagulation status fall into the broad categories of diagnostics, monitoring and drug discovery and are described further below in detail. As a brief summary, in some embodiments, diagnostics through use of waveform analysis can include, for example:
diagnosis of and/or screening for coagulation disorders (e.g., bleeding or hypercoagulation)
discrimination between different coagulation factor deficiencies or between discrete levels of coagulation factors
diagnostics or predictions of treatment methods by studying effects of adding one or more coagulation factors to a plasma sample of a patient such as factor VIII (“FVIII”), factor IX (“FIX”), and factor FVII (“FVII”)
discrimination between hemophiliac plasma, with and without inhibitors, and with or without therapeutic proteins used to treat hemophilia, such as differences in type of hemophilia
discrimination between different activators of coagulation tests, such as, for example, differences between silica and ellagic acid
   In one or more embodiments, monitoring through use of waveform analysis can include, for example:
monitoring to determine whether and/or the extent to which one or more therapeutic agents can be used successfully in a patient
monitoring tailored for patient-specific therapies and/or therapeutic dosing
   In one or more embodiments, drug discovery through use of waveform analysis can include, for example:
screening for new therapeutic compounds to treat coagulation blood disorders
screening for agents to reverse specific therapies, e.g. overdose of anticoagulant therapy
screening for a dosage and/or efficacy of new anticoagulants or procoagulants

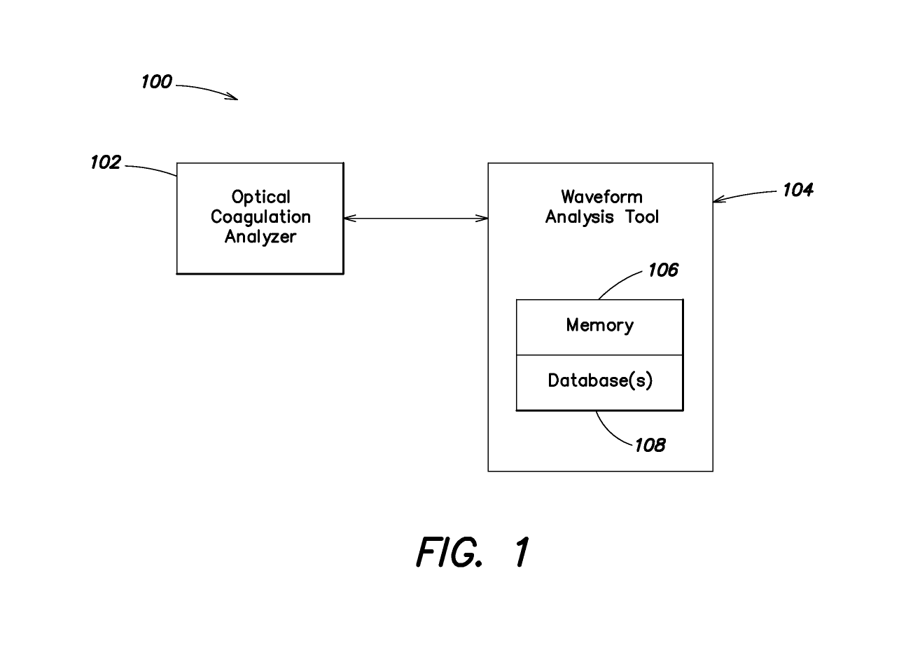

Systems and methods for carrying out these and other embodiments are described below with reference to FIGS. 1-14.

### System for Using Waveform Analysis

FIG. 1 is a schematic diagram of an example system 100 for performing waveform analysis on coagulation data in accordance with certain embodiments. With reference to FIG. 1, the system 100 includes an optical or other coagulation analyzer 102 coupled to a waveform analysis tool 104. The waveform analysis tool 104 includes a memory 106 and one or more databases 108. Alternatively, the waveform analysis tool 104 can interface with one or more local or remote databases which can provide a data storage component that the tool 104 can access for analysis. The waveform analysis tool 104 can be part of the optical coagulation analyzer 102 or separate from the optical coagulation analyzer 102 as shown in FIG. 1. While not shown in FIG. 1, the waveform analysis tool 104 can include input/output devices such as a display, keyboard, mouse, as well as other components.

In the embodiment of FIG. 1, the coagulation analyzer 102 is an optical coagulation analyzer. The optical coagulation analyzer 102 can be any suitable optical coagulation analyzer, such as the ACL TOP® available from instrument Laboratory, Bedford, Mass. or the Siemens BCS-XP available from Siemens Healthcare Diagnostics, Tarrytown, N.Y. Other optical coagulation analyzers can be used. The wavelength of light used by the optical coagulation analyzer 102 can be any suitable wavelength, and/or any number of wavelengths. In some embodiments, the wavelength(s) employed by the optical coagulation analyzer 102 can be about 650-671 nanometers or greater. For example, the wavelength can be chosen as one that has no or little interference from hemoglobin or bilirubin (e.g., is not absorbed substantially by hemoglobin or bilirubin).

The waveform analysis tool 104 can be implemented in hardware, software or a combination thereof. In some embodiments, the optical coagulation analyzer 102 can be modified to include the functionality of the waveform analysis tool 104. For example, the optical coagulation analyzer 102 can include computer program code for further processing data collected by the coagulation analyzer 102. In other embodiments, the waveform analysis tool 104 can be a standalone computer such as a desktop computer, laptop computer, tablet computer, electronic laboratory book such as a BioBook available from IBDS of Chicago, Ill., having computer program code for carrying out one or more of the methods described herein.

In certain embodiments, the waveform analysis tool 104 can receive data output from the optical coagulation analyzer 102. The data from the optical coagulation analyzer 102 can be exported to a program such as Microsoft Excel or MS Access available from Microsoft Corporation of Redmond, Wash., executing on the waveform analysis tool 104. In some embodiments, the data from the optical coagulation analyzer 102 can be subjected to waveform analysis using a program such as Sigmaplot available from Systat Software Inc., San Jose, Calif.

This system 100 can be used, for example, to perform one or more embodiment methods described below.

In certain example embodiments, raw data can be exported from a lab device (e.g., coagulation analyzer 102) and imported to an electronic laboratory book such as a BioBook available from IBDS or the like (that serves as waveform analysis tool 104). For example, this can be done by a BioBook import definition. The time range can be the range beginning from the first time index without a “D” and end by the last time index. For each data block, a summary section is available at the end of the raw data file, with information about the concentration and clot point of the data block. The concentrations can be imported out of the summary section of the file. Sample Name can be imported from the summary section of the file if available. The clot point can be imported out of the summary section.

Once the data is in the BioBook, a chart of the data generated by waveform analysis can be plotted, displaying all samples at once and/or on a Plot chart with the respective baseline by sample (if desired). The data can then be manipulated to manually overwrite the concentration and/or to enter a multiplier for the time points to calculate different time periods.

An electronic laboratory book, computer, or the like can compute the derived results, for example:

| | | | Time to | | | | | | | |
|---|---|---|---|---|---|---|---|---|---|---|
| | | Time | Peak | | | | | | | |
| | Sample | to | for | AUC | AUC | Slope | | lysis | | Clot |
| | Name | Peak | slope | 0-inf | t1-t2 | T lag | slope | T_lag | _slope | Point |
| 1 | 801 | 277 | 510 | 50650.019 | 47306.046 | 252 | 2.55663 | 479.0 | −0.370 | 866 |
| 2 | 802 | 267 | 472 | 19889.652 | 18732.871 | 246 | 1.962744 | 563.0 | −0.182 | 421 |

After that data is obtained, in some embodiments, one or more of the following can be performed, if desired:
Derived Result Dose Response Curve Analysis;
Query for specific Sample Derived Results with Dose;
Generate EC50/IC50 data and Dose Response Curves;
Derived Result Publishing to Pix;
After the Derived Results Analysis, publish to Pix for data.

### Waveform Analysis Methods

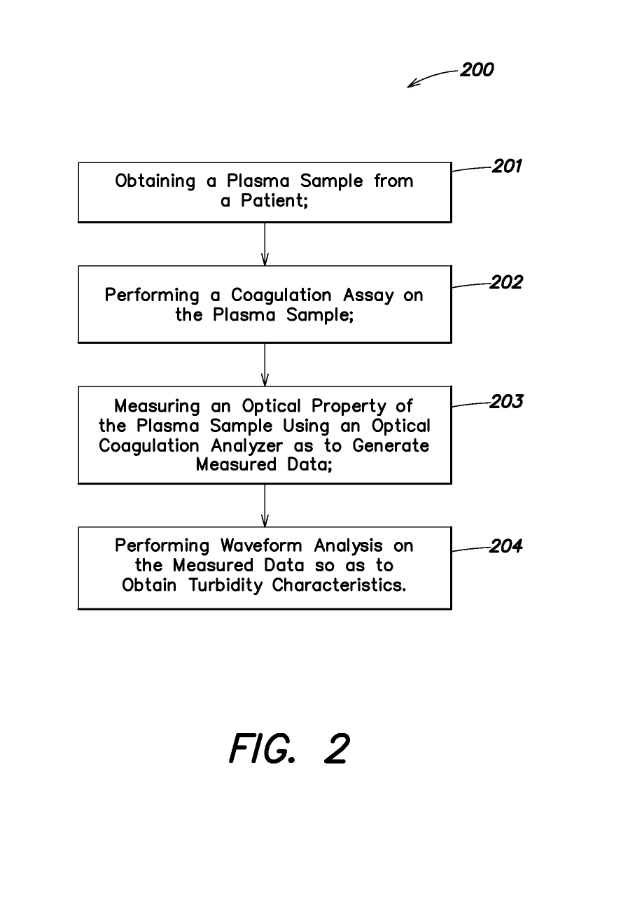

FIG. 2 illustrates a flowchart of an example method 200 for waveform analysis in accordance with certain embodiments. With reference to FIG. 2, method 200 begins with Block 201 in which a plasma sample is obtained from a patient.

The patient can be, for example, any animal subject, including a human or a non-human subject. Any plasma sample can be used. Plasma can be obtained from a patient by any method known in the art, including by first drawing the patient's blood and preparing plasma from the blood sample by separating blood cells from plasma. Plasma can also be obtained from commercial sources, such as, for example, factor-deficient (for example, including one or more of factor VIII, factor IX, factor VII, von Willebrand factor, factor XI, and factor XII) plasma from individual or pooled individual donors (HRF, Inc., Raleigh, N.C.; George King Biomedical, Overland Park, Kans.), normal control (Instrumentation Laboratory, Orangeburg N.Y.), special test control 2 (Instrumentation Laboratory, Orangeburg, N.Y.), and/or calibration plasma (Instrumentation Laboratory, Orangeburg, N.Y.).

In Block 202, a coagulation assay is performed on the plasma sample. In some embodiments, the coagulation assay is aPTT, PT, a diluted prothrombin time (“dPT”) assay, a factor-specific assay. However, any coagulation assay can be used that can be performed using an optical coagulation analyzer or other suitable coagulation analyzer. An aPTT assay car: be performed using, for example, APTT-SP kit (Instrumentation Laboratory, Orangeburg, N.Y.) or SynthAFax® kit (Instrumentation Laboratory, Orangeburg, N.Y.). In certain embodiments, depending on the coagulation assay, the plasma can be activated so that a clot can form (e.g., using silica, celite, ellagic acid or the like).

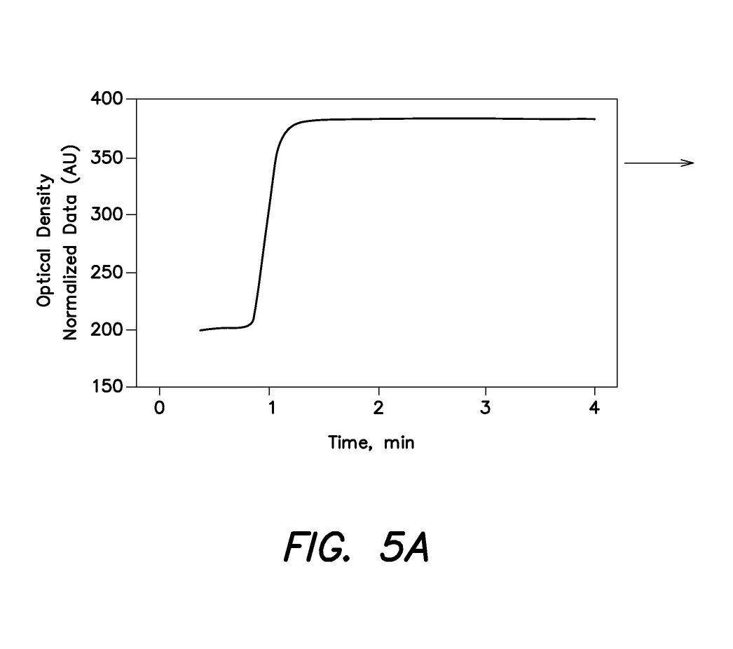

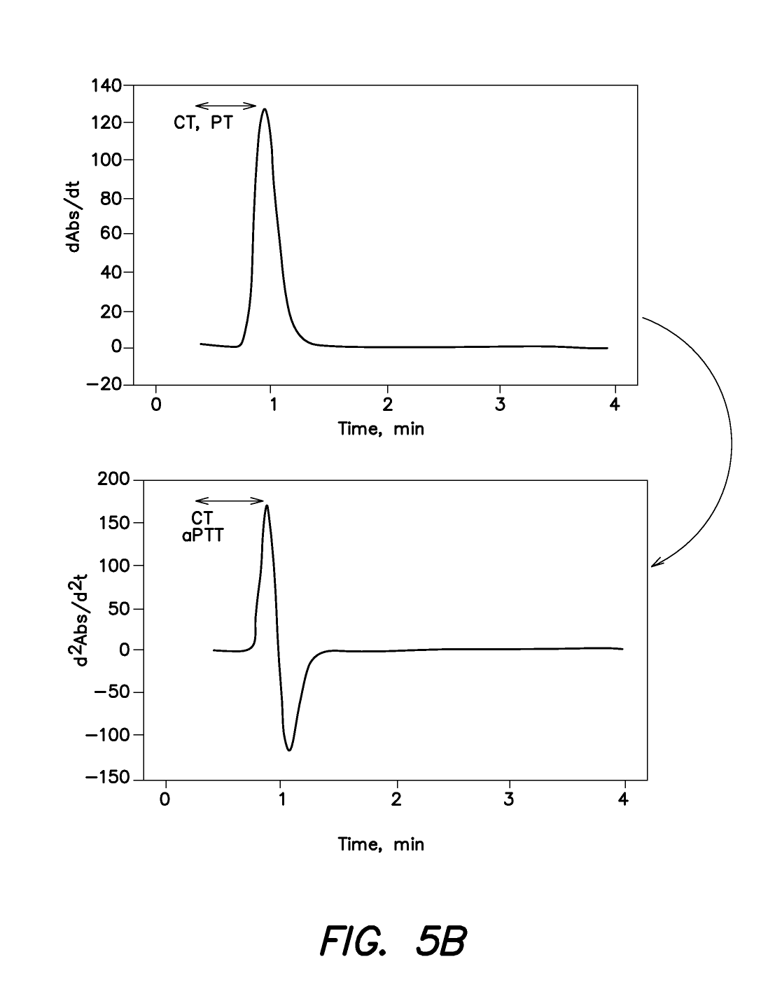

In Block 203, a property of the plasma sample is measured using the coagulation analyzer 102 so as to generate measured data. The property can include turbidity and/or optical density, for example. In some embodiments, the clot formation (of fibrin) can be observed by measuring the optical density or turbidity over time using the coagulation analyzer 102, thereby generating the measured data. (FIG. 5A illustrates optical density change versus time in accordance with some embodiments as described below.)

In Block 204, waveform analysis is performed on the measured data using the waveform analysis tool 104. For example, waveform analysis can encapsulate the analysis of the turbidity data, and the first derivative and second derivative transforms of the turbidity data. Further quantitative descriptors of the data from the turbidity, its first derivative transform (rate of coagulation), and its second derivative transform (acceleration of coagulation), or both, can be performed. These quantitative descriptors of the waveforms can include maxima (peak), minima, slope, area under the curve (AUC) and/or the like. As stated, collection, manipulation, transformation and/or other analyzing processes performed on data measured by a coagulation analyzer are referred to collectively herein as waveform analysis.

In some embodiments, waveform analysis can assess the kinetics of coagulation. Currently, coagulation is typically expressed in terms of clot time CT, the time required to develop a defined increase in optical density in optical analyzers. The kinetics of coagulation can provide information on the perturbation of specific parts of the coagulation system, resulting in different rates and extents of coagulation and consequent turbidity development.

As will be described further below, in certain embodiments, waveform analysis can be performed using the waveform analysis tool 104 to obtain coagulation status information for a plasma sample. Clot time and clotting kinetics of coagulation reactions are examples of coagulation status. As detailed below, waveform analysis can also allow, for example, diagnosing and monitoring of bleeding disorders, patient status and discovery of new therapeutic compounds of coagulation blood disorders, etc.

### Method for Correlating Disease States of Hemophilia a Patients

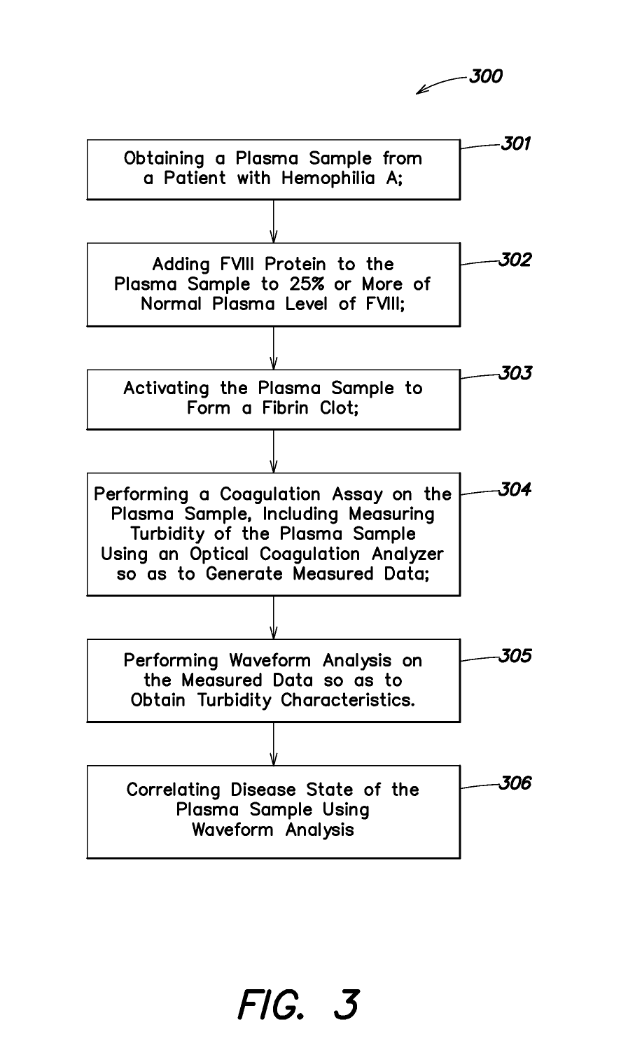

FIG. 3 illustrates a flowchart of a method 300 in accordance with certain embodiments to correlate different disease states of hemophilia A patients. With reference to FIG. 3, method 300 begins with Block 301 in which a plasma sample from a patient with hemophilia A is obtained. The patient can be any animal subject, including a human or a non-human subject. Plasma can be obtained from a patient by any method known in the art, including by first drawing the patient's blood and preparing plasma from the blood sample by separating blood cells from plasma.

In Block 302, FVIII protein (also referred to as an “FVIII spike”) is added to the plasma sample to simulate a pre-defined FVIII level in the plasma. Any level of FVIII protein may be simulated by adding the appropriate amount of FVIII protein to the plasma sample. In one example embodiment, FVIII protein is added to the plasma sample to about 25% or more of a normal plasma level of FVIII.

In Block 303, the plasma sample is activated to form a fibrin clot (e.g., using silica, celite, ellagic acid or the like).

In Block 304, a coagulation test is performed on the plasma sample. In some embodiments, the coagulation assay can be aPTT, PT, or a factor-specific assay. However, any coagulation assay can be used that can be performed using an optical coagulation analyzer or other suitable coagulation analyzer. An aPTT assay can be performed using, for example, APTT-SP kit (Instrumentation Laboratory, Orangeburg, N.Y.) or SynthAFax® kit (Instrumentation Laboratory, Orangeburg, N.Y.). In some embodiments, the coagulation assay can include measuring turbidity of the plasma sample using an optical coagulation analyzer 102, to generate measured data. The clot formation (of fibrin) can be observed by measuring the optical density and/or turbidity over time using the optical coagulation analyzer 102, thereby generating the measured data. (See, for example, FIG. 5A.)

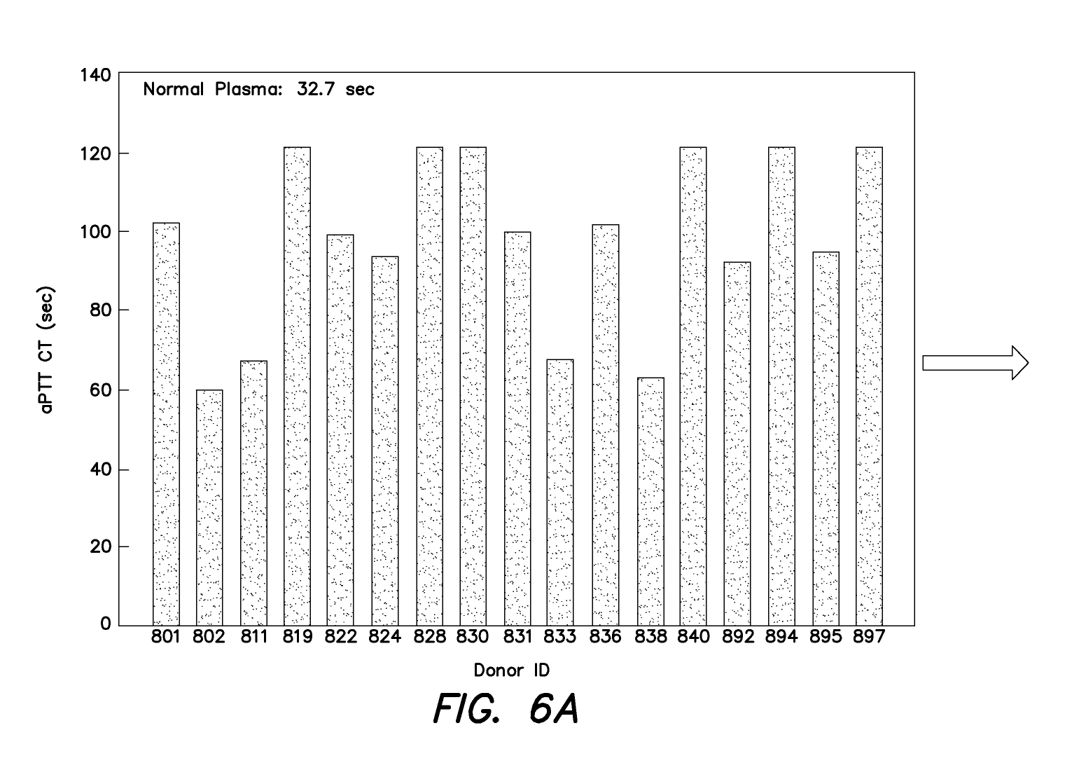

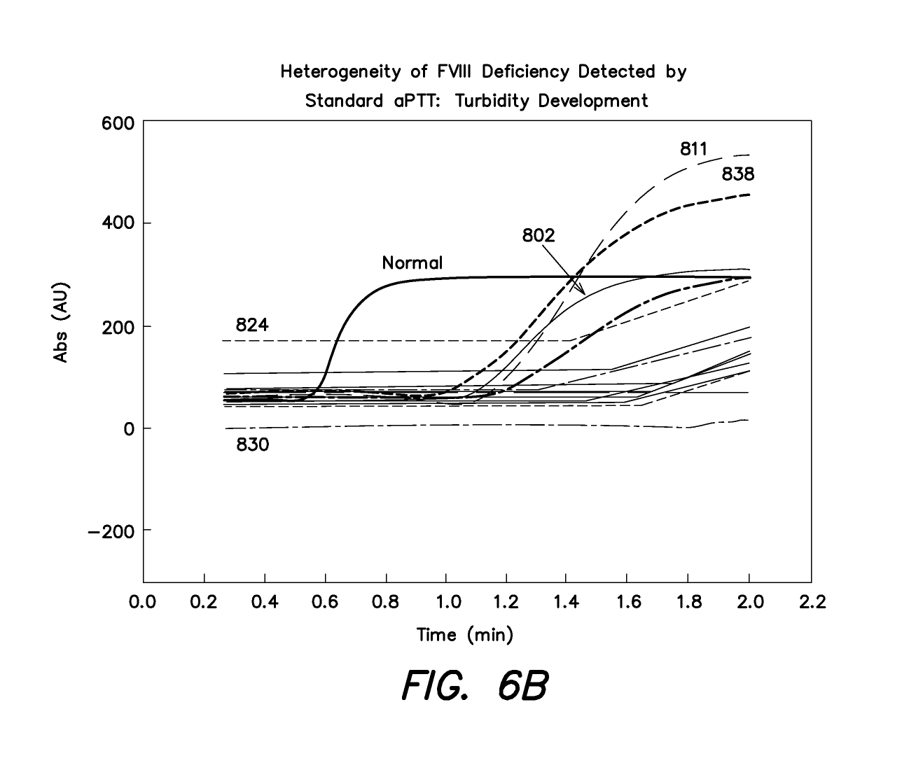

In Block 305, waveform analysis is performed on the measured data using the waveform analysis tool 104. For example, the first and/or second derivative of measured density and/or turbidity over time can be computed to reveal rate of coagulation and/or acceleration of coagulation (see, for example, FIG. 5B). Plots of coagulation rate and/or coagulation acceleration for different levels of FVIII can be computed and displayed together to identify trends (see, for example, FIG. 6A), or the like.

In Block 306, the disease state of the plasma sample of the patient can be correlated to defects in the plasma using the waveform analysis. As stated, first and second derivatives of density and/or turbidity over time, comparison of multiple plots of various coagulation data, and/or the like can reveal coagulation processes and mechanisms not observable from conventional clot time data.

In certain embodiments, the method 300 can further include obtaining one or more of fibrinogen level and thrombin level in the plasma sample and correlating disease state of the plasma sample using waveform analysis and/or one or more of the fibrinogen level and the thrombin level. Fibrinogen level and thrombin level can be determined by any method in the art. The plasma samples of different hemophilia A patients will likely have different coagulation statuses, as can be determined through use of the methods described herein.

### Method for Determining Coagulation Status in Response to Coagulation Factor(s)

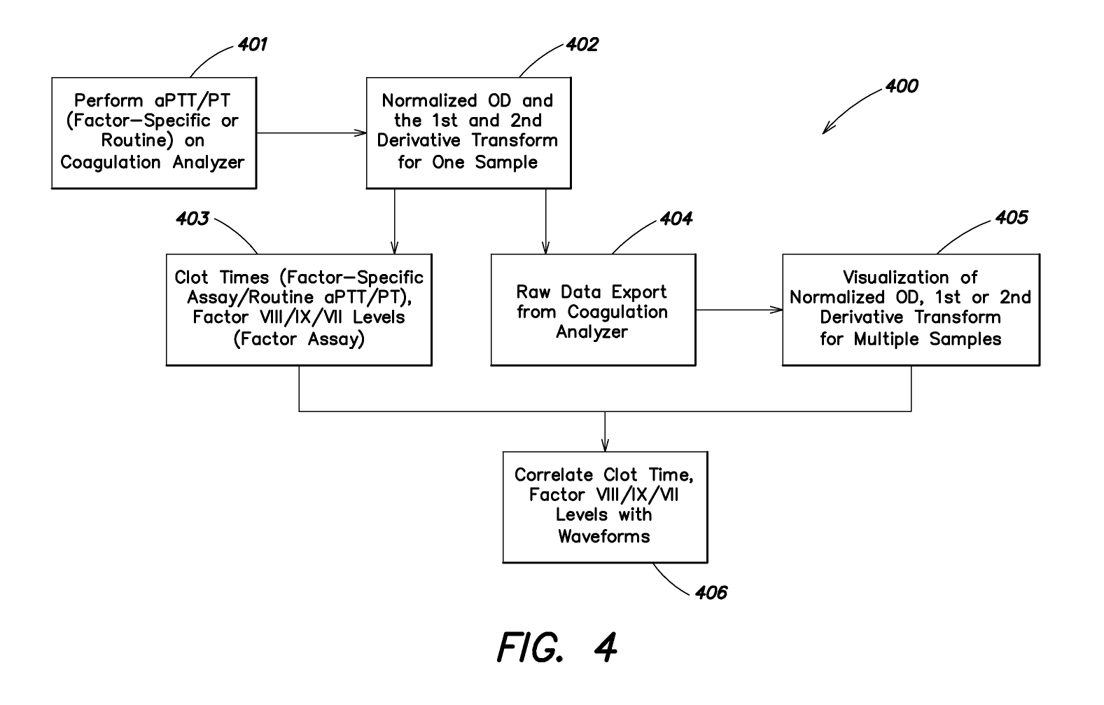

FIG. 4 is a flow diagram illustrating a method 400 for using waveform analysis to determine coagulation status in response to one or more coagulation factors. With reference to FIG. 4, in Block 401, a factor-specific or routine coagulation assay is performed, such as aPTT/PT, on a coagulation analyzer (e.g., optical coagulation analyzer 102).

In Block 402, normalized optical density (OD) and first and second derivative data are output from the coagulation analyzer, or computed from data measured by the coagulation analyzer (e.g., using waveform analysis tool 104). (See, for example, FIGS. 5A-5B.)

In Block 403, clot times and factor levels are obtained. Clot times can be obtained directly from the coagulation analyzer (assuming no test failures), and factor levels can be measured and/or known.

In Block 404, raw data is exported from the coagulation analyzer and imported to the waveform analysis tool 104.

In Block 405, waveform analysis is performed on the raw data from the coagulation analyzer. For example, normalized optical density, first and second derivative transforms, area under each curve, etc., for multiple samples may be collected, plotted, correlated and/or otherwise analyzed.

In Block 406, clot times, factor levels and waveforms are correlated to establish relationships between clot time and factor levels. Such information can include, for example, clotting dynamics, coagulation response to factors, and the like.

Through use of waveform analysis, coagulation analyzer data that would otherwise be used primarily for clot time measurements can be used to gain insights into coagulation diagnostics, clotting dynamics, coagulation monitoring, drug discovery, and the like (generally referred to as “coagulation status”). Specific examples are provided below.

### EXAMPLES

### Application of Waveform Analysis for Individualized Patient Assessment and Patient Care

### Example 1

### Example Waveform Analysis Methods

FIGS. 5A and 5B illustrate example data that can be generated and/or analyzed by waveform analysis using the waveform analysis tool 104. The data includes normalized absorbance data, as well as first and second derivative data. Other data can be generated and/or analyzed. FIG. 5A displays optical density (“OD”) change vs. time from data obtained from optical analyzer 102. The top graph of FIG. 5B shows a plot of the first derivative transform of the measured data from FIG. 5A. The bottom graph of FIG. 5B shows a plot of the second derivative transform of the data from the top graph of FIG. 5B.

### Example 2

### Application of Waveform Analysis for Improved Accuracy of Individual Plasma Testing and Potential Treatment

Although waveform analysis can be applied to any plasma sample, hemophilia A plasma was chosen as a specific example, primarily due to the heterogeneity responses obtained. This heterogeneity is evident in the variable CT obtained with stand-alone aPTT results (FIG. 6A). The heterogeneity of HemA plasma is even more apparent by waveform analysis and the description of the turbidity characteristics of individual plasma (FIG. 6B). Turbidity characteristics can include turbidity maximum, rate of turbidity development and/or acceleration of turbidity development.

As described below, the application of waveform analysis enables greater accuracy of coagulation testing. For example, in many cases, waveform analysis can provide clot time (CT) for coagulation assays. Table 1 provides a list of plasma samples that were tested for clot times using an optical coagulation analyzer (identified under “Instrumentation” heading of Table 1). CT provided by the vendor is compared to measured CT. As can be seen from Table 1, CT could not be measured for several samples as a result of failed aPTT assays (e.g., samples 819, 828, 830, 840, 894 and 897 failed to produce clot times).

TABLE 1
Stand-Alone aPTT Results for Hemophilia A Donors

| | Instrumentation, Kit | CT Provided | CT |
|---|---|---|---|
| Sample | Used for Vendor aPTT | by Vendor, s | Determined, s |
| Special test 2 | ACL TOP ®, aPTT-SP | N/A | 78.7 |
| Normal control | ACL TOP ®, aPTT-SP | 24.7-32.7 | 28.5 |
| Normal rep 1 | ACL TOP ®, aPTT-SP | 30.5 | 32.9 |
| Normal rep 2 | ACL TOP ®, aPTT-SP | | 33.1 |
| Normal rep 3 | ACL TOP ®, aPTT-SP | | 32.1 |
| 801 (cross | ACL TOP ®, aPTT-SP | 92 | 102.3 |
| reactive | | | |
| material | | | |
| negative) | | | |
| 802 | ACL TOP ®, aPTT-SP | 54 | 59.4 |
| 811 | ACL TOP ®, aPTT-SP | 75 | 66.7 |
| 819 | ACL TOP ®, aPTT-SP | 101 | FAILED |
| 822 | ACL TOP ®, aPTT-SP | 80 | 98.8 |
| 824 | ACL TOP ®, aPTT-SP | 92 | 93.2 |
| 828 | ACL TOP ®, aPTT-SP | 104 | FAILED |
| 830 | ACL TOP ®, aPTT-SP | 110 | FAILED |
| 831 | ACL TOP ®, aPTT-SP | 94 | 99.4 |
| 833 | ACL TOP ®, aPTT-SP | 65 | 66.9 |
| 836 | ACL TOP ®, aPTT-SP | 92 | 101.1 |
| 838 | ACL TOP ®, aPTT-SP | 59 | 62.5 |
| 840 | ACL TOP ®, aPTT-SP | 80 | FAILED |
| 892 | Stago, Tcoag Activator | 81 | 91.7 |
| 894 | Stago, Tcoag Activator | 82.5 | FAILED |
| 895 | Stago, Tcoag Activator | 74.5 | 94.3 |
| 897 | Stago, Tcoag Activator | 75.9 | FAILED |

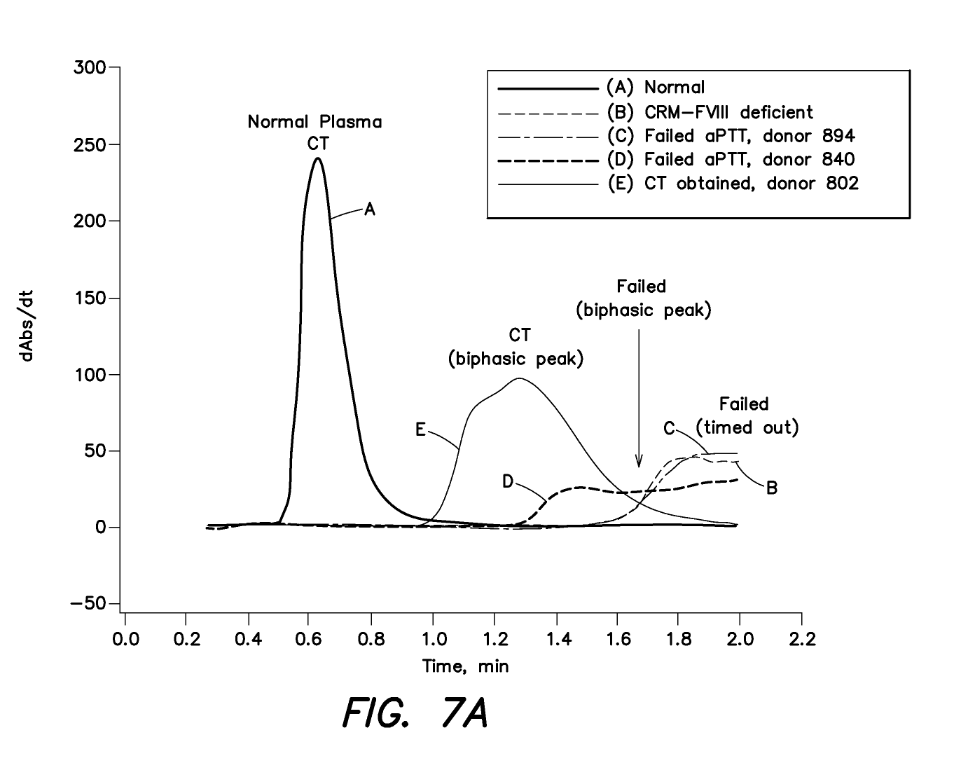

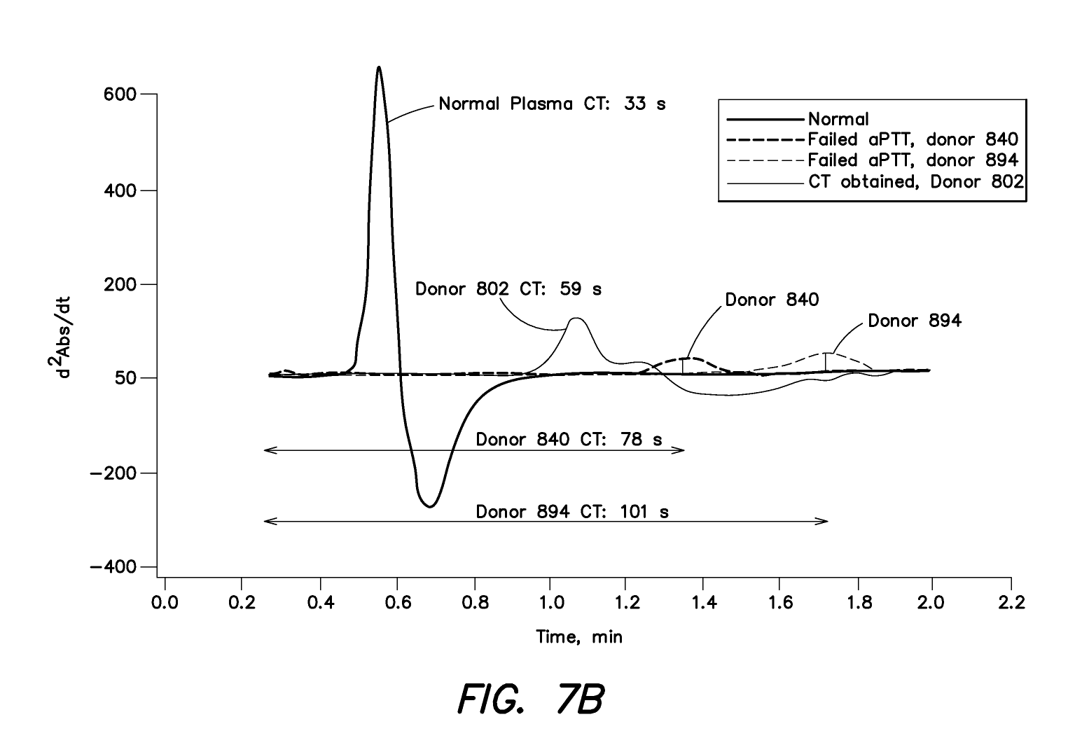

Waveform analysis of the turbidity data from the coagulation analyzers allowed analysis of coagulation kinetics for the failed assays and identification of reasons for the failures to obtain CT times. For example, FIG. 7A illustrates first derivative waveform profiles of problematic aPTT results obtained from optical analyzer 102 (donors 840 and 894). Normal plasma samples and one hemophilia A plasma sample that yielded clot time (CT) values are shown in FIG. 7A for reference (donor 802). Waveform analysis of individual samples identified plasma that did not clot, as well as samples that failed to produce clot times due to CT determination errors.

CT determination errors were associated with biphasic rate profiles (first-derivative waveforms). CT result failures can occur when the sample first- or second-derivative curves do not meet the analyzer software criteria for a valid curve (e.g., such as by “timing out”). For example, plasma sample from donor 802 passed the criteria and yielded CT values, but plasma sample from donor 840 did not.

FIG. 7B illustrates waveform analysis employing second derivative transformation data of the data from FIG. 7A in accordance with certain embodiments. With reference to FIG. 7B, visualization of the acceleration profiles (second-derivatives) of measured coagulation data allows CT extrapolation. For example, aPTT CT can be derived from time needed to reach maximum acceleration. Maximum acceleration can be determined visually or by determining the maximum in the second-derivative column of the data export of the coagulation analyzer (if available). As shown in FIGS. 7A and 7B, waveform analysis indicates that the most common cause of failure in hemophilia A plasma testing by aPTT is the slow coagulation rate. Table 2 illustrates extrapolated CT of plasma failing to yield aPTT results. Waveform analysis was employed to obtain the extrapolated results (See Table 2 below and FIGS. 7A and 7B).

TABLE 2
Extrapolated CT of Plasma Failing to Yield aPTT Results

| | Vendor-provided | CT by aPTT | CT aPTT + Waveform |
|---|---|---|---|
| Sample | CT, s | Alone, s | Analysis, s |
| 819 | 101 | FAILED | 191.1 (CT determined |
| | | | using an extended |
| | | | assay and WA) |
| 828 | 104 | FAILED | 157.7 (CT determined |
| | | | using an extended |
| | | | assay and WA) |
| 830 | 110 | FAILED | 110.5 |
| 840 | 80 | FAILED | 77.7 |
| 894 | 82.5 | FAILED | 100.5 |
| 897 | 75.9 | FAILED | 90.7 |

As shown above, combined aPTT and waveform analysis offers several advantages over stand-alone aPTT. CTs can be assigned to samples that failed stand-alone aPTT results. The enhanced ability to assign CTs to samples can translate to reduced retesting and to higher laboratory throughput. Additionally, waveform analysis, in combination with the CT provided by stand-alone aPTT, can provide additional information on the relative rates and acceleration of OD change (e.g., coagulation).

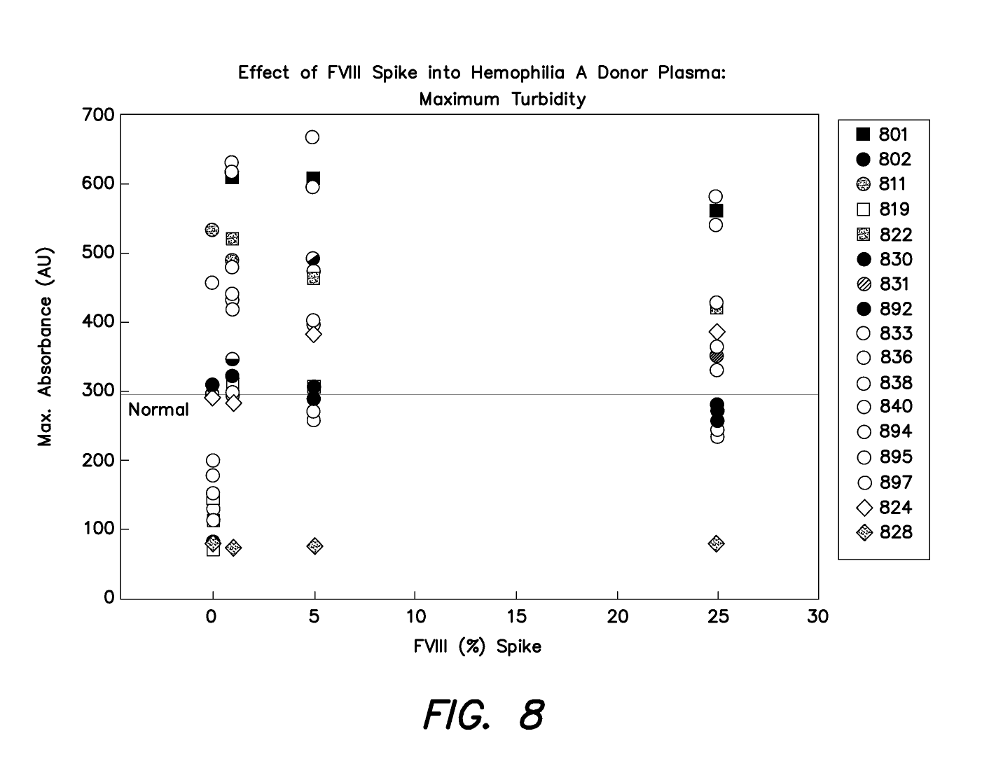

Turbidity characteristics obtained by combining coagulation assay results and waveform analysis can be used to broadly assess hemophilia A patient response to FVIII therapy. For example, FIG. 8 illustrates waveform analysis of turbidity maxima versus FVIII level in hemophilia A plasma samples. FIG. 8 illustrates that measuring turbidity maxima of hemophilia A plasma samples containing FVIII at different levels results in segregation of the plasma samples into distinct turbidity maxima groups, suggesting different and variable clot quality in response, depending on the FVIII level present. In the embodiment shown in FIG. 8, maximum turbidity response to 25% FVIII (e.g., the level deemed sufficient to promote “normal” coagulation) can be divided into four clusters. The different clusters could indicate differential clot structure formation, especially as turbidity characteristics have been related to fibrin structures (Weisel et al., “COMPUTER MODELING OF FIBRIN POLYMERIZATION KINETICS CORRELATED WITH ELECTRON MICROSCOPE AND TURBIDITY OBSERVATIONS: CLOT STRUCTURE AND ASSEMBLY ARE KINETICALLY CONTROLLED,” (1992) Biophys. J. 63: 11-128).

As described in more detail below, in some embodiments, correlation of turbidity characteristics to individual hemophilia A plasma response to rFVII using coagulation assay and waveform analysis indicated:
Hemophilia A plasma response can be divided into two subpopulations
The major (about 67%) subpopulations appeared to have turbidity changes consistent with reduced thrombin generation secondary to their FVIII defect
a minor subpopulation appeared to have some additional defect(s) that reduced their maximal rates of turbidity development

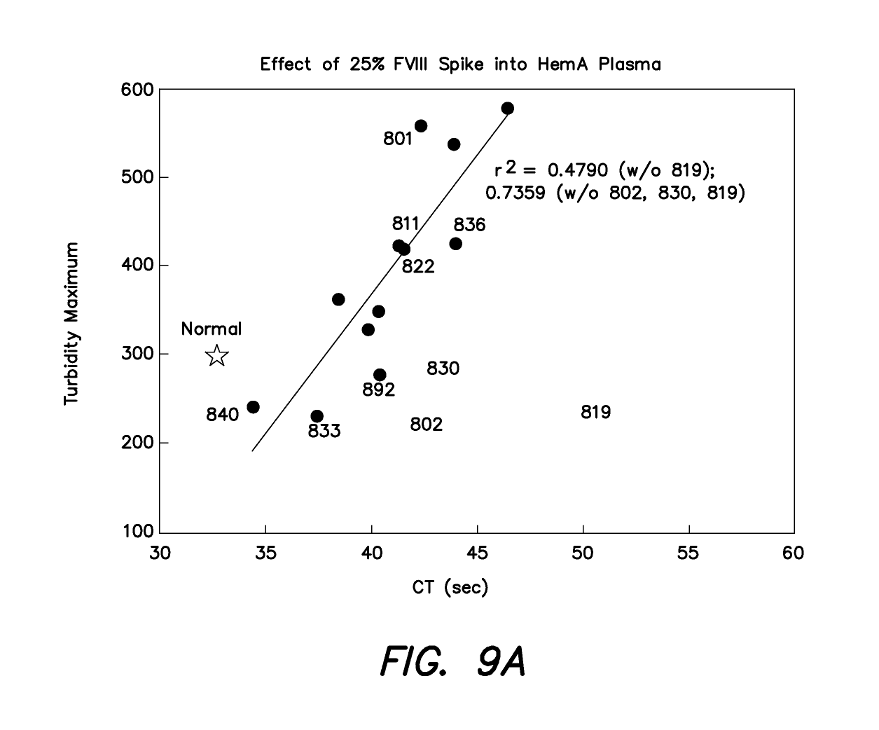

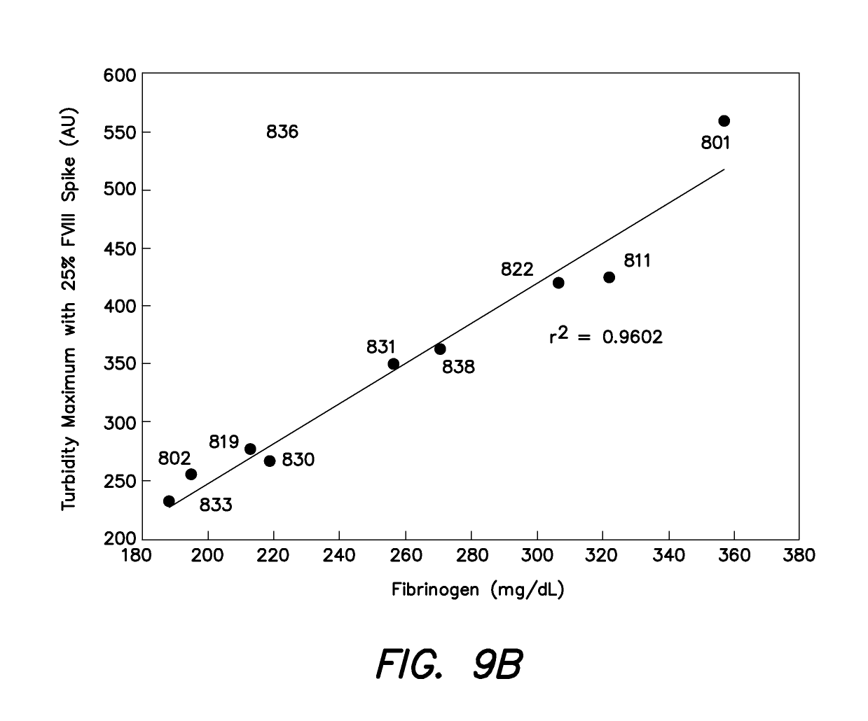

Comparison of the turbidity maxima with clot time, the time needed to achieve a defined peak acceleration, indicated roughly 2 types of responses to coagulation initiation. FIG. 9A, a plot of turbidity maxima versus clot time CT, allows assessment of the turbidity characteristics of individual plasma containing 25% FVIII, the minimum level of FVIII needed to achieve near-normal coagulation (e.g., no bleeding except after challenge). Compared to normal plasma, all hemophilia A (“HemA”) plasma had longer clot time values and, for the most part, larger turbidity maxima.

The bulk of the responses (67%) for the HemA plasma showed a linear relationship between turbidity and CT. The remainder of the plasma responses showed longer CT compared to the turbidity maxima. The turbidity changes seen in the majority of the plasma tested are consistent with the reduced thrombin generation characteristic of FVIII deficiency. In a subgroup of the plasma tested (donors 819, 802, 830, and 833), the turbidity maxima were lower than expected from their prolonged CT (see, for example, the CT results of Tables 1 and 2 compared with the results of FIG. 9A; note symbols were removed from “outlier” samples). Since turbidity change is related to fibrin polymerization, these plasma samples may have a fibrin defect in addition to their FVIII deficiency. Fibrinogen level assessment indicated that the plasma in the minor subgroup had fibrinogen levels which fall into the low end of normal (see

TABLE 3

| ID | Fibrinogen (mg/dL) |
|---|---|
| 801 | 356.9481 |
| 802 | 194.9596 |
| 811 | 322.1048 |
| 819 | 213.0258 |
| 822 | 306.821 |
| 830 | 219.0182 |
| 831 | 256.5672 |
| 833 | 188.4599 |
| 835 | 310.5228 |
| 836 | 205.465 |
| 838 | 270.6502 |
| 841 | 229.6769 |
| 842 | 207.3113 |
| normal | 256.5672 |

In contrast to the turbidity maxima and maximum rate of turbidity development, the maximal acceleration data attained with HemA containing 25% FVIII was consistent with the FVIII deficiency (see FIG. 9C which is a plot of Maximum Acceleration of Turbidity Development versus CT). As shown in FIG. 9C, FVIII amplifies and accelerates coagulation physiologically.

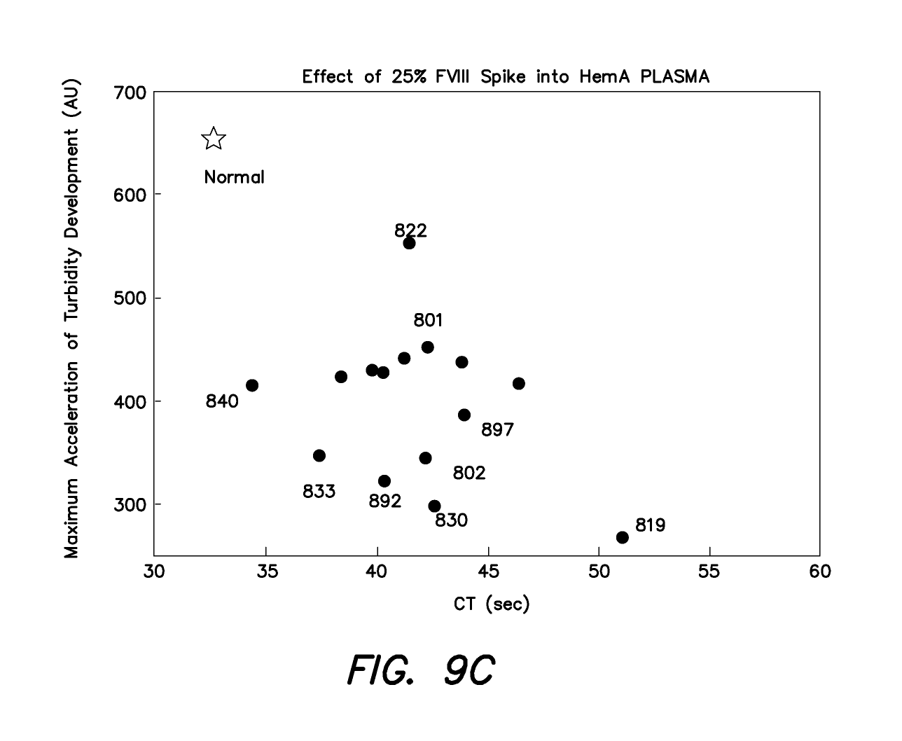

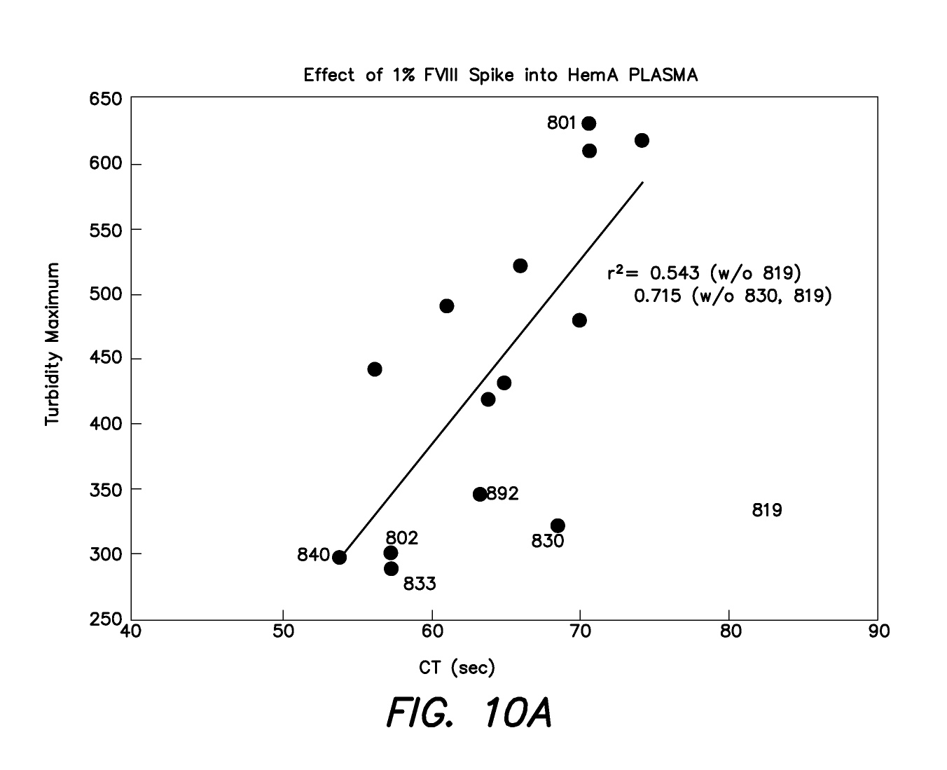

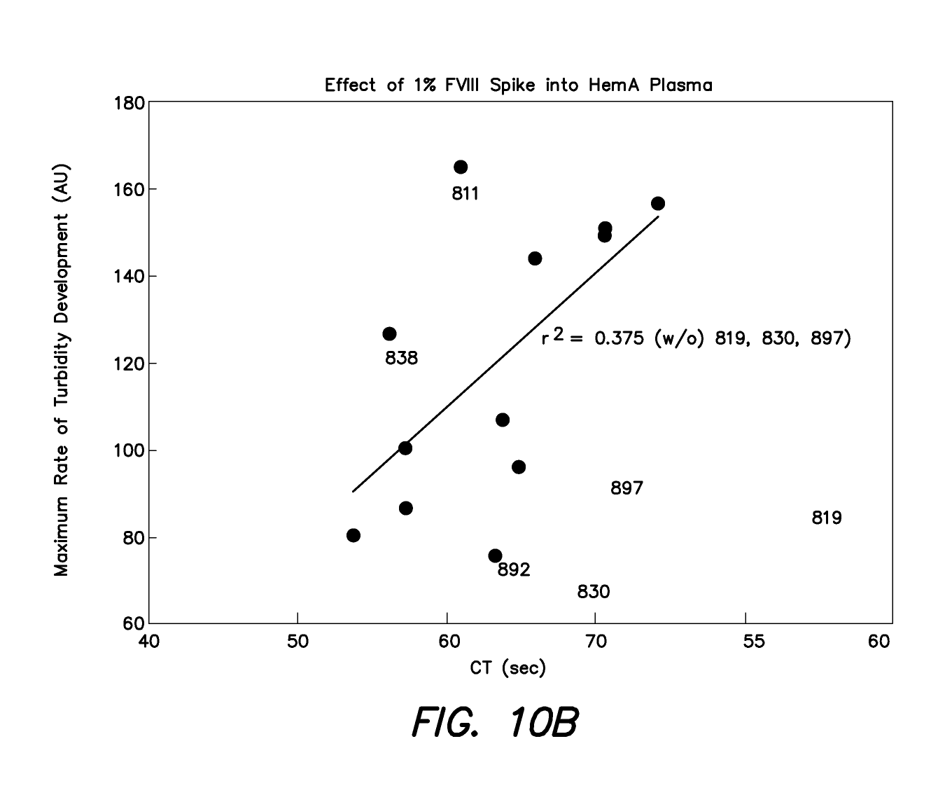

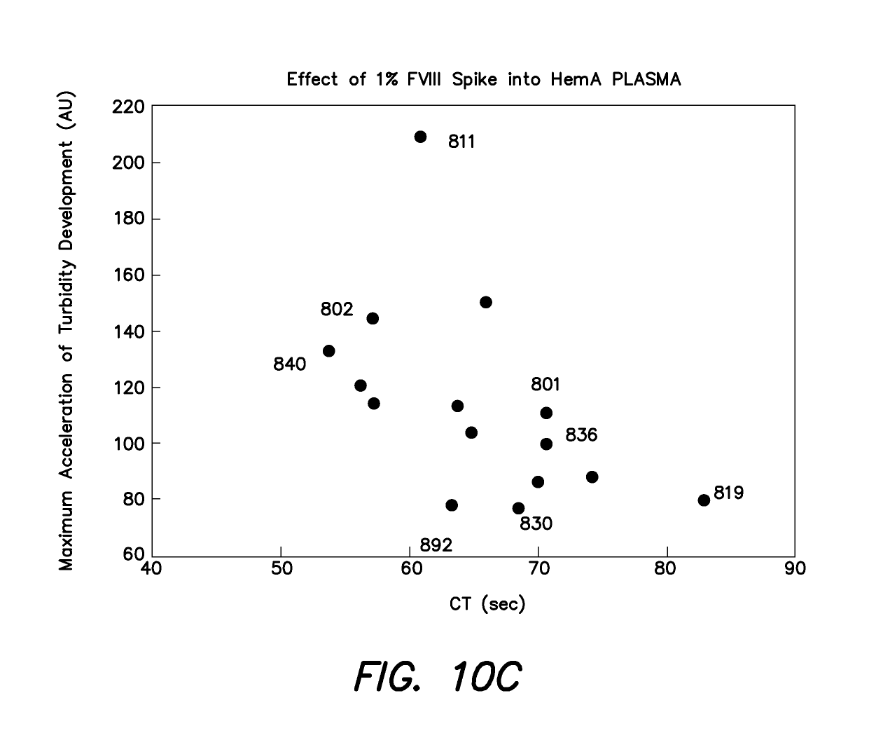

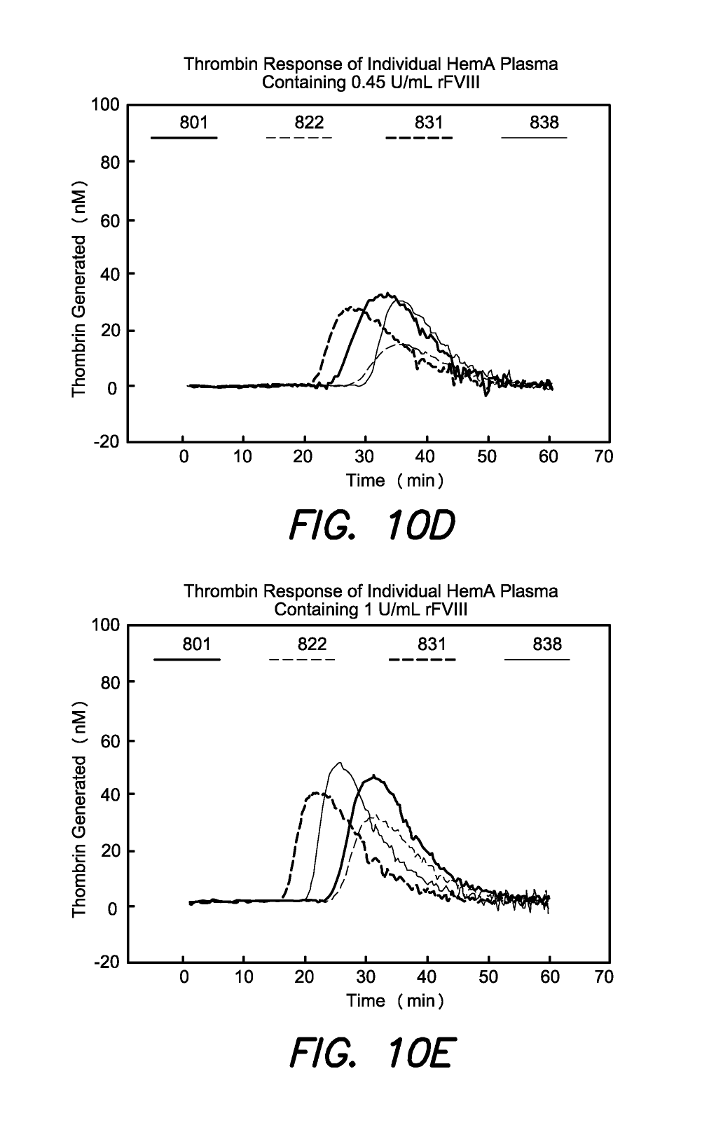

Application of waveform analysis to HemA plasma containing 1% FVIII, the level below which the frequency of spontaneous bleeding increases, yielded similar results analogous to those obtained with plasma containing 25% FVIII (See, for example, FIGS. 10A-10C).

Comparison of the turbidity responses of the HemA plasma versus normal plasma indicated clear deficiencies in the HemA plasma. Improving HemA coagulation response decreased the CT, reduced the turbidity maxima and increased the acceleration of turbidity development. Therefore, waveform analysis can be applied to individualized patient dosing or treatment.

The premise behind FVIII therapy is replacement of a functional deficiency in a cofactor employed for coagulation, such as thrombin generation and fibrin polymerization. In FIGS. 10D-E, the thrombin generation response of some hemophilia A plasma to different levels of FVIII was monitored over time. FIGS. 10D-E show the thrombin response of plasma containing 1% FVIII (per the vendor HRF Inc.) from the indicated hemophilia A donors. FVIII (0.45 U/mL in FIG. 10D and 1 U/mL in FIG. 10E) was added to each donor plasma, and coagulation was initiated with 1 pM TF-4 μM PL (PPP-Low). Thrombin generation was monitored using a fluorogenic substrate assay for thrombin, as described by the manufacturer (Stago).

Without the addition of FVIII, the hemophilia A plasma tested had minimal, baseline thrombin response, consistent with the reduced (≦1%) FVIII levels of the plasma. With the addition of FVIII, the thrombin responses of the plasma tested showed dose-dependent shortening of the onset and increased peak thrombin responses. Interestingly, the hemophilia A plasma tested demonstrated individual variations in thrombin responses, suggesting that this can translate to individual variations in fibrin polymer properties, e.g., ability of the drug (rFVIII) to restore coagulation to the same extent in individual hemophilia A patients. On the basis of these thrombin generation results, individual variations in turbidity characteristics can be anticipated.

Waveform analysis can be very informative when coupled with patient correlative data such as the type or brand of FVIII usage and/or the presence of potential joint issues. Other relevant correlative patient data can include the quantity of FVIII used/dose, the frequency of dosing, the age of diagnosis and/or development of target joints, the frequency of bleeding despite adequate plasma FVIII levels, and/or the like, as shown in Table 4 below.

TABLE 4
Example of Potential Correlative Patient Data

| | | | | | | | Total FVIII |
|---|---|---|---|---|---|---|---|
| patient | treatment | washout | | | | JOINT | Antigen (% |
| ID | info | period | GENDER | AGE | RACE | ISSUES | Normal) |
| HRF 801 | RECOMBINANT | 7 DAYS | MALE | 63 YRS | WHT | HIPS | Undetectable |
| | | | | | | AND | |
| | | | | | | ANKLES | |
| HRF 802 | RECOMBINANT | 7 DAYS | MALE | 53 YRS | WHT | HIPS | 3.90 |
| | | | | | | AND | |
| | | | | | | ANKLES | |
| HRF 811 | ADVATE | 7 DAYS | MALE | 49 YRS | WHT | ELBOW | 109.68 |
| | | | | | | AND | |
| | | | | | | KNEES | |
| HRF 819 | RECOMBINANT | 7 DAYS | MALE | 45 YRS | BLK | KNEES | 1.67 |
| HRF 822 | RECOMBINANT | 7 DAYS | MALE | 35 YRS | BLK | KNEES | 1.48 |
| | | | | | | AND | |
| | | | | | | ANKLES | |
| HRF 824 | rFVIIa | 7 DAYS | MALE | 36 YRS | BLK | KNEES | 0.80 |
| HRF 828 | rFVIIa | 7 DAYS | MALE | 37 YRS | BLK | KNEES | Undetectable |
| | | | | | | AND | |
| | | | | | | HIPS | |
| HRF 830 | RECOMBINANT | 7 DAYS | MALE | 27 YRS | BLK | NONE | Not Done |
| HRF 831 | RECOMBINANT | 7 DAYS | MALE | 28 YRS | BLK | NONE | 0.77 |
| HRF 833 | RECOMBINANT | 7 DAYS | MALE | 22 YRS | WHT | NONE | 1.06 |
| HRF 835 | RECOMBINANT | 7 DAYS | MALE | 33 YRS | WHT | NONE | plasma not |
| | | | | | | | purchased |
| HRF 836 | RECOMBINANT | 7 DAYS | MALE | 23 YRS | WHT | NONE | 0.6332174 |
| HRF 838 | RECOMBINANT | 7 DAYS | MALE | 22 YRS | WHT | NONE | 1.28 |
| HRF 840 | ADVATE | 7 DAYS | MALE | 26 YRS | WHT | NONE | Not Done |
| HRF 841 | RECOMBINANT | 7 DAYS | MALE | 19 YRS | WHT | NONE | plasme not |
| | | | | | | | purchased |
| HRF 842 | RECOMBINANT | 7 DAYS | MALE | 29 YRS | BLK | ELBOWS | plasma not |
| | | | | | | AND | purchased |
| | | | | | | KNEES | |

| | | |
|---|---|---|
| GK 892 | No Data Available | Not Done |
| GK 894 | | Not Done |
| GK 895 | | Not Done |
| GK 897 | | Not Done |

### Example 3

### Application of Waveform Analysis to Facilitate Drug Discovery

Waveform analysis can be applied to drug discovery by identifying additional procoagulant drugs, identifying potential mechanisms of procoagulant action and by detecting procoagulant drug reversal in emergency treatment of patients anticoagulated with FXa- or thrombin-specific inhibitors.

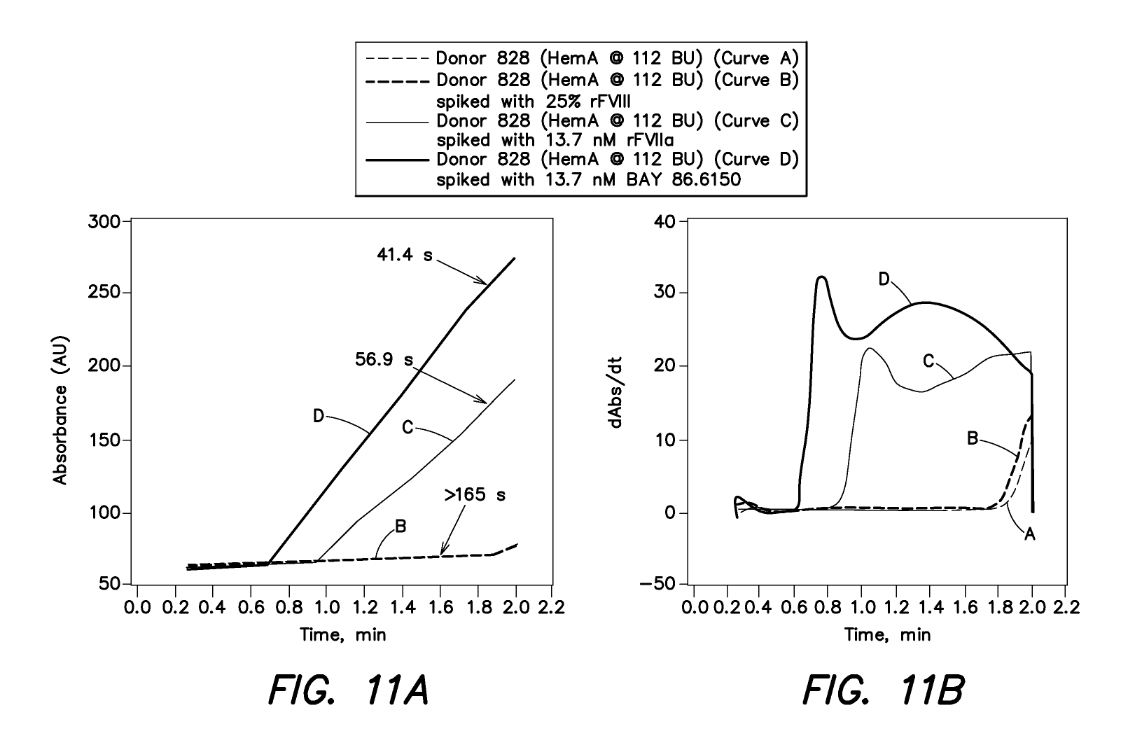

FIGS. 11A and 11B are plots of optical absorbance and the first derivative transformation of optical absorbance versus time, respectively, for HemA plasma samples with 112 BU treated with various procoagulants including rFVIII, rFVIIa, and BAY86-6150. FIGS. 11A and 11B show the procoagulant response of hemophilia A plasma containing a high level of inhibitors (anti-drug antibodies) in a standard aPTT assay. Waveform analysis allows the discrimination of effective drug treatment for these inhibitor patients. For example, the ineffectiveness of rFVIII is evident (see curve “B” in FIG. 11A and FIG. 11B). The use of rFVIIa to restore coagulation to these patients is evident in curve “C” (FIGS. 11A and 11B). Further, the greater effectiveness of BAY 86-6150 over rFVIIa for hemophilia with inhibitors is evident in the shorter lag times for the absorbance waveform (FIG. 11A) and its first derivative transform (FIG. 11B). The higher maximum rate of OD increase obtained for BAY 86-6150 versus rFVIIa (FIG. 11A) suggests more rapid coagulation. Absorbance and/or first derivative data for each plasma sample can be stored, for example, in database 108 of waveform analysis tool 104 (FIG. 1). Thus, waveform analysis can provide significant information regarding the relative effectiveness of procoagulant drugs.

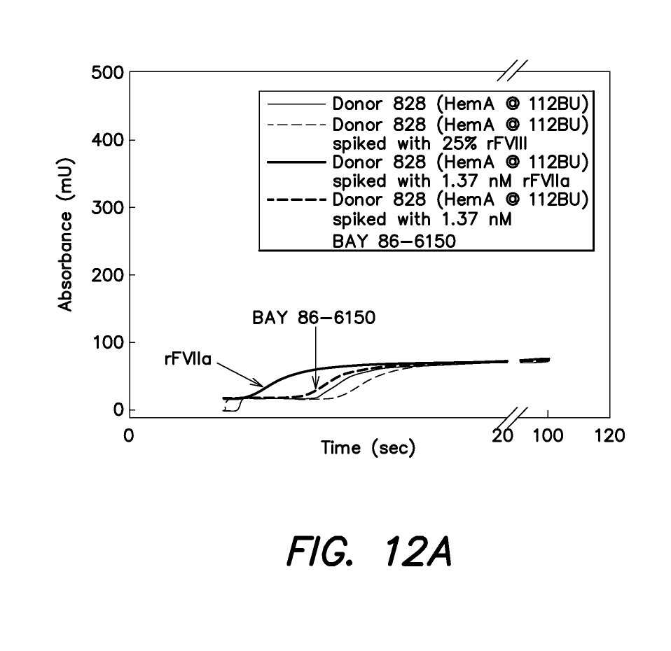

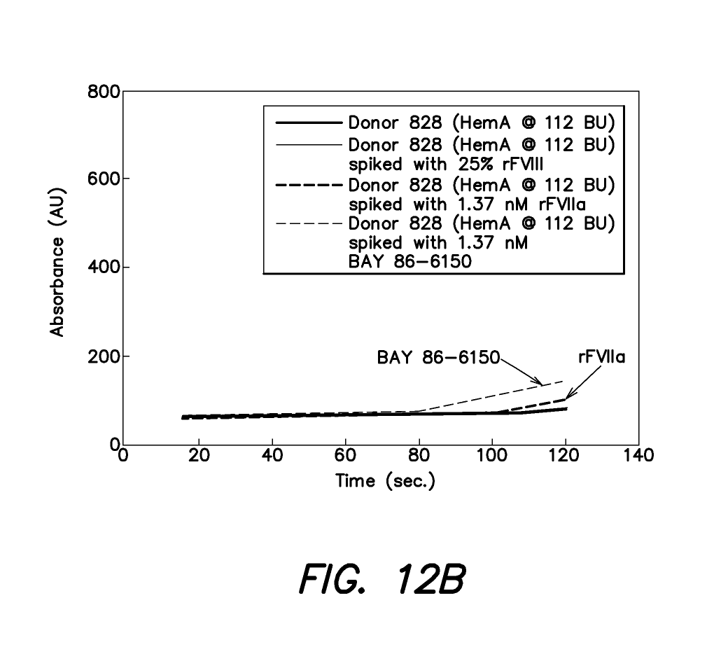

In FIGS. 12A-12B, waveform analysis was applied to demonstrate potential differences in mechanism of procoagulant action. Performance of rFVIIa and BAY 86-6150 in hemophilia A plasma with inhibitors was assessed by PT (FIG. 12A) and aPTT (FIG. 12B), followed by waveform analysis. The PT assesses the extrinsic part of coagulation, and is sensitive to FVII levels, which are normal in hemophilia A patients, without and with inhibitors. Therefore, the turbidity maxima obtained for hemophilia A plasma with inhibitors were low, regardless of whether the procoagulant present was rFVIII, rFVIIa or BAY 86-6150. However, the presence of rFVIIa and BAY 86-6150 shortened the onset of coagulation in the PT assay. In the embodiment shown, the onset of coagulation in the PT was faster with rFVIIa than with BAY 86-6150. In contrast to the PT, hemophilia A plasma with inhibitors containing rFVIII had a poor coagulation response (as in FIG. 11A, curve “B”). Unlike the coagulation response with the PT, rFVIIa had a slower onset in the aPTT than the BAY 86-6150. These results indicate that rFVIIa mediates coagulation in hemophilia A plasma primarily by the extrinsic portion of the coagulation assay while the coagulation mediated by BAY 86-6150 may be mediated more by the intrinsic portion of coagulation. Finally, as rFVIIa has been associated with thrombotic risks, the difference in mechanism of action could have potential safety implications.

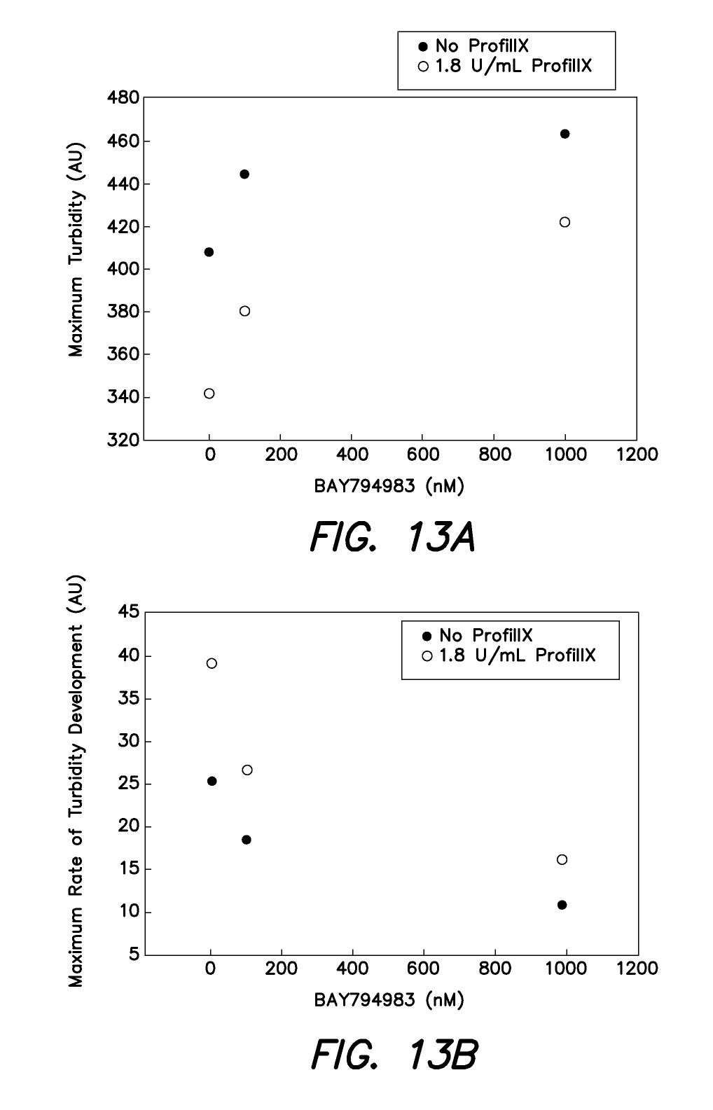

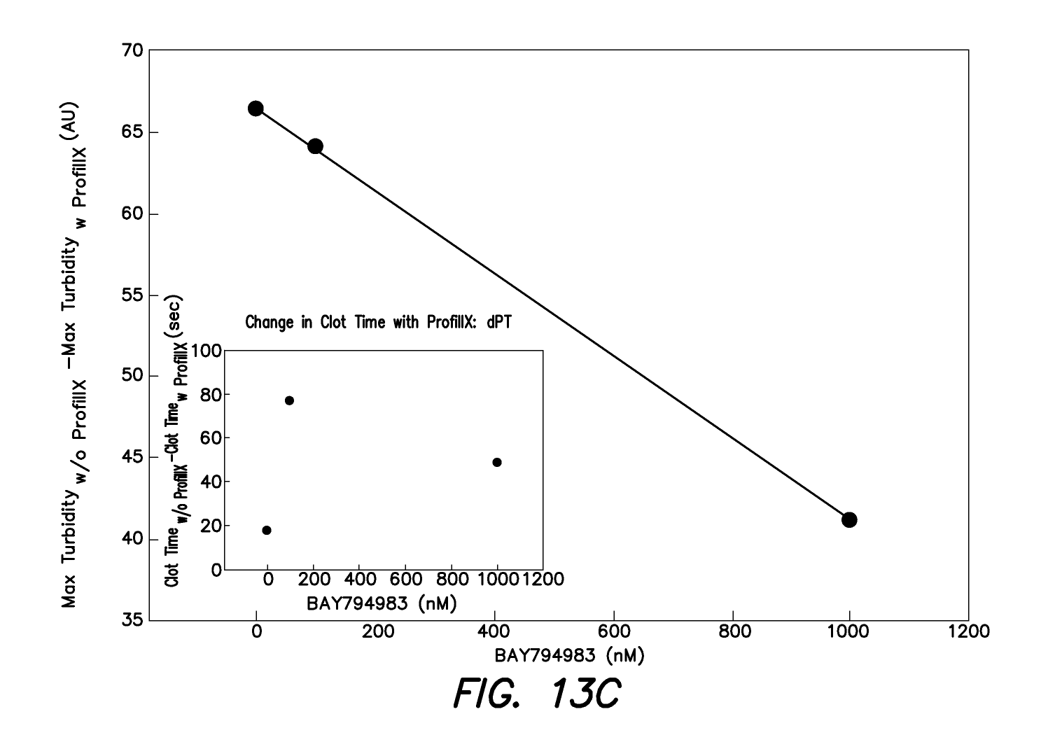

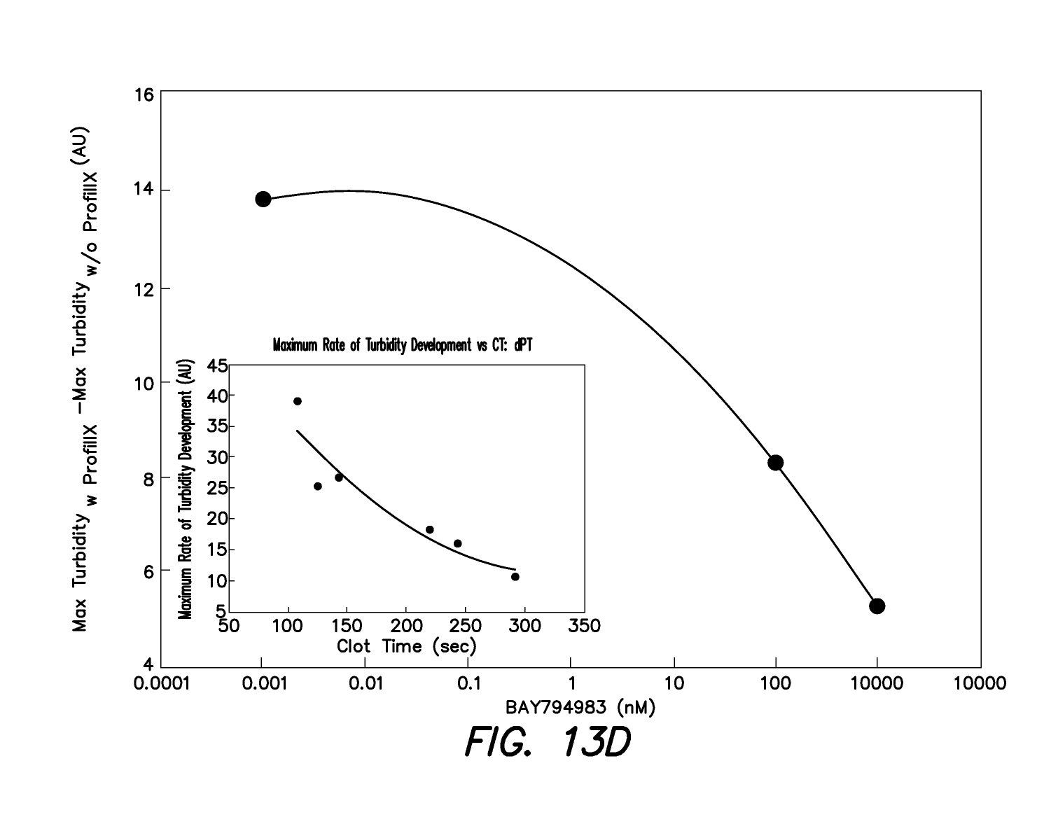

The application of waveform analysis to detect reversal of anticoagulants is shown in FIGS. 13A-13D. While the anticoagulant example depicted here is a research molecule BAY 794983, a FXa-specific inhibitor, waveform analysis can potentially be applied to study the action of the FXa- or thrombin-specific anticoagulants. These new classes of anticoagulants were designed to obviate the need for patient monitoring. However, it may be desirable to reverse the action of anticoagulants such as BAY 794983 in some emergency situations.

The effect of BAY 794983 (0-1000 nM) on normal plasma coagulation was assessed by dilute PT (dPT) assay. The dPT assay was performed by diluting the tissue factor activator Recombiplastin, to increase the ability to discriminate both anticoagulation and the reversal of anticoagulation. Reversal of anticoagulation was performed with a fixed concentration (1.8 U/mL) of a commercially available procoagulant ProfilIX. Parallel samples with buffer addition served to assess the effect of the anticoagulant alone.

As shown in FIG. 13A, BAY 794983 induced prolongation of the clot time and increased turbidity maxima in a dose-dependent manner. The presence of ProfilIX reduced the prolongation in clot time and reduced the increase in turbidity maxima induced by the anticoagulant. Consistent with its reversal of BAY 794983, ProfilIX increased the maximal rate of turbidity development (FIG. 13B). Plots of the difference in the turbidity maxima (FIG. 13C) or maximal rate of turbidity change (FIG. 13D) without and with ProfilIX indicated a dose-dependent response with BAY 794983. This indicates that the ability of ProfilIX to reverse BAY 794983 anticoagulation depends on the inhibitor concentration present in the plasma. In contrast to the dose-dependent differential in turbidity characteristics, the difference in CT appears to show no clear dose-dependent response with BAY 794983 (FIG. 13C, inset). However, clot time did relate to maximal rate of turbidity (FIG. 13D, inset).

Therefore, waveform analysis has been applied to detect the reversal of anticoagulant therapy, and depending on the coagulation assay used, the duration of the test can be as short as 2 minutes for standard aPTT or PT assay or as long as 15 minutes for an assay such as the dPT. Transfer of the optical density data and the subsequent waveform analysis performed as described above is consistent with the timeframe needed to guide emergency care.

The above shows that, in certain embodiments, methods employing waveform analysis can be used to assess procoagulant activity. Any test compound can be similarly analyzed. For example, a test compound can be substituted for ProfilIX and procoagulant activity tested as described above.

### Example 4

### Application of Waveform Analysis to Diagnosis of Coagulation Disorder

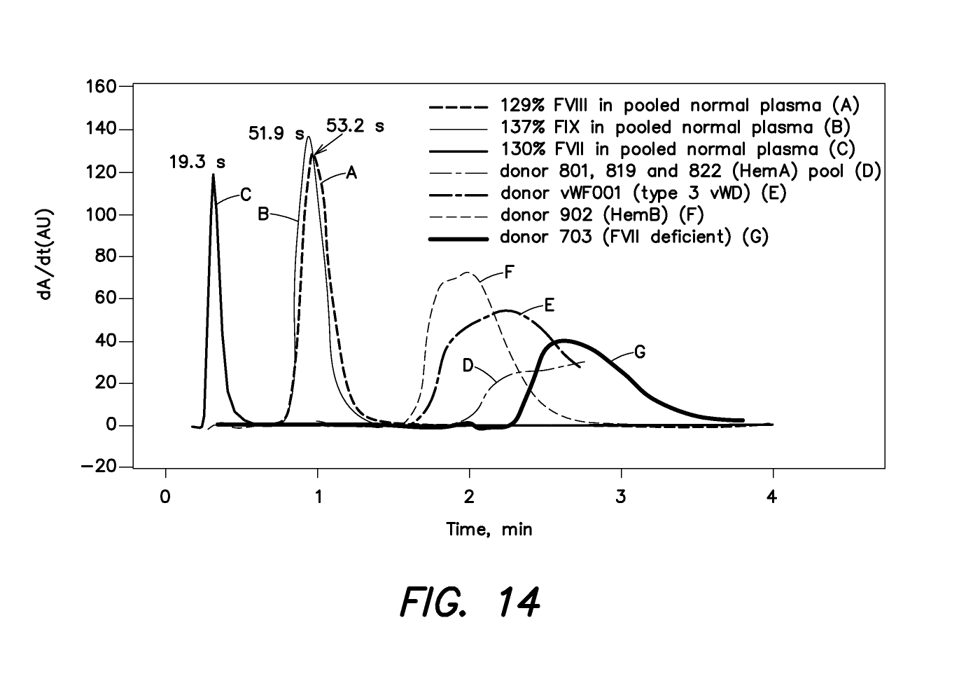

In certain embodiments, waveform analysis can be used in combination with a factor-specific assay to diagnose which blood coagulation factor is deficient in a patient. Waveform analysis of coagulation data for plasma from factor deficient (such as, for example, FVIII, von Willebrand factor (“vWD”), FIX, and FVII) donors can show both absorbance and first-derivative waveform differences (FIG. 14). Differences in first-derivative curves for severe hemophilia A plasma relative to von Willebrand factor type III plasma with 5% residual FVIII activity were observed. The hemophilic plasma had a longer lag time (about 113 vs. 93 seconds, respectively) and lower peak amplitude (about 29.7 vs. 54.7 absorbance unit (“AU”)) than the von Willebrand factor type III plasma. The control reactions containing>1.00% FVIII, FIX, and FVII had considerably shorter CT (indicated in FIG. 14) and much higher maximal rates of turbidity development compared to the deficient plasma. Table 5 below and FIG. 14 illustrate waveform analysis characterstics for specific coagulation factor-mediated coagulation. Specifically, waveform analysis in accordance with certain embodiments allows detection of FVIII, FIX, FVII and vWD (type 3) deficiency (via first derivative transformation of clot time data from an optical coagulation analyzer). Superposition of individual first derivative waveform transforms indicates (1) coagulation can be more rapid in FIX-, FVIII- and FVII-deficiency; and (2) type 3 vWD plasma (which also is FVIII-deficient) can be distinguished from hemophilia A plasma.

TABLE 5
Factor-Deficient Plasma Tested by Factor-
Specific Assay and Waveform Analysis

| | | Assay | Retested Assay |
|---|---|---|---|
| Plasma | Factor Assay | Activity (%) | Activity (%) |
| Donors 801 + | FVIII aPTT | 0.2 | 0.2 |
| 819 + 822 pool | | | |
| (hemophilia A) | | | |
| Donor vWF001 | FVIII aPTT | 2.5 | |
| (type 3 vWD) | | | |
| Donor 902 | FIX aPTT | 1.5 | |
| (hemophilia B) | | | |
| Donor 703 (FVII | FVII PT | 0.2 | No clot |
| deficient) | | | |

### Other Applications of Waveform Analysis

As described above, optical density, turbidity and/or other data measured for a coagulation assay by a coagulation analyzer can be collected, manipulated, transformed and/or otherwise analyzed to obtain additional insights into coagulation processes and/or coagulation mechanisms (e.g., as part of waveform analysis).

### Diagnostic Methods

In some embodiments, application of waveform analysis for diagnosis can be performed by performing factor-specific coagulation assays or standard aPTT with a variety of activators, including silica activators. Samples can range from purified proteins to plasma samples. As described below, these methods are able to diagnose blood disorders accurately and reliably.

Seventeen hemophilia A donor plasma samples were subjected to aPTT on the ACL TOP® coagulation analyzer, followed by waveform analysis of the raw data, exported from the ACL TOP® coagulation analyzer. The samples were obtained from donors who had been subjected to factor-specific and Bethesda assays. One donor was cross-reactive material (antigen)-negative, and two had FVIII inhibitors at approximately 2 and approximately 110 BU. With the exception of one donor who had slightly higher FVIII activity levels (1%-2%), the bulk of the hemophilia A plasma contained<1% FVIII levels.

Raw data was exported from the optical coagulation analyzer, and subsequent data manipulations were performed using Microsoft Excel or MS Access. Data manipulations included conversion of read intervals to time intervals, and organization of data into groups to be compared. Visualization and comparisons of the waveforms were performed using Sigmaplot, which also permits verification of aPTT clot times by identifying the time required to achieve maximum acceleration of coagulation. Other software code and/or programs can be employed for waveform analysis.

The aPTT results were initially assessed without the benefit of waveform analysis. The aPTT failed to yield a clot time in 40% of the samples, although the re-test decreased the failure rate by an additional 50%. Application of waveform analysis, which allowed visualization of coagulation kinetics, indicated that aPTT failure was attributable to prolongation of the clot time beyond the assay duration or to altered coagulation kinetics and consequent clot time determination errors.

In one or more embodiments, a method is provided to diagnose bleeding disorders in a patient, by obtaining a coagulation status of a plasma sample of the patient using waveform analysis. For instance, the method can be employed to screen a patient suspected of having coagulation abnormality (e.g., bleeding or hypercoagulation) or a patient undergoing surgery. The coagulation status obtained can be compared to the coagulation status of other plasma samples, such as a normal plasma sample.

In certain embodiments, a diagnostic method is provided to discriminate between different coagulation factor deficiencies or between discrete levels of coagulation factors by determining a coagulation status of the plasma sample of a patient using waveform analysis. This method can distinguish coagulation reactions resulting, for example, from defects in different aspects of the coagulation cascade even if the clotting times are the same, because the kinetics of coagulation reactions (an example of coagulation status) can be observed using waveform analysis. Therefore, the method can diagnose which coagulation factors are missing or reduced in a patient's plasma using waveform analysis. A lack or a reduction of different factors can be distinguished by the waveform analysis.

In certain embodiments, a diagnostic method is provided by adding one or more coagulation factors to the plasma sample of a patient prior to performing the coagulation assay to simulate the effect of specific plasma coagulation factor levels on the coagulation status of the plasma sample, using waveform analysis. The coagulation status obtained can be compared to the coagulation status of other plasma samples, such as a normal plasma sample. Restoration of coagulation status data to that or near that of normal plasma can determine which factor was defective. The coagulation factor can be in the intrinsic or in the extrinsic pathway and include, for example, one or more of factor VIII (“FVIII”), factor IX (“FIX”), and factor FVII (“FVII”).

In certain embodiments, a diagnostic method is provided to discriminate between hemophiliac plasma, with and without inhibitors, and with or without therapeutic proteins used to treat hemophilia, by determining a coagulation status of a plasma sample using waveform analysis. Waveform analysis can distinguish these various differing circumstances, including differences in type of hemophilia, such as for example, hemophilia A vs. hemophilia B, with or without inhibitors, and with or without containing therapeutic proteins used to treat hemophilia in the plasma.

In certain embodiments, methods are provided that can discriminate between different activators of coagulation, such as, for example, differences between silica and tissue factor due to their differential ability to activate clotting factors and thus their differential ability to activate plasma deficient in specific factors, using waveform analysis.

### Monitoring Methods

In certain embodiments, a monitoring method is provided to determine whether one or more therapeutic agents can be used successfully in patient. The one or more therapeutic agents (including, for example, procoagulant or anticoagulant) can be added to a patient's plasma sample and a determination can be made whether the added therapeutic agent changed a coagulation status of the plasma sample using data including waveform analysis. The coagulation status determined with the added therapeutic agent can be compared to the coagulation status of other plasma samples, such as, for example, a coagulation status of plasma without the added therapeutic agent.

Because individual responses to therapy vary, allowing more tailored or patient specific therapies and/or therapeutic dosings is highly beneficial both in terms of effectiveness of clotting factors and the underlying costs. Adding back different levels of procoagulants or anticoagulants to the patient's plasma and monitoring individual patient response using waveform analysis allows patient-specific responses to be monitored and better calibration of doses.

In certain embodiments, another method is provided to determine which therapeutic agent is efficacious for a patient. Such a method can include, for example, dividing the plasma sample of a patient into several portions or fractions (aliquots) of the sample, adding either none or one or more therapeutic agents to a sample of that plasma, and determining whether the added agent changed the coagulation status of the test plasma sample using waveform analysis. For example, a patient suffering from hemophilia can have his or her plasma tested by adding different amounts of recombinant factor VIII to the plasma and then performing the methods described herein.

In certain embodiments, a method is provided to determine the efficacious dose of a therapeutic agent. Such a method can include, for example, dividing the plasma sample of a patient into several portions or fractions (aliquots), adding none or a different dosage of a therapeutic agent or a different combination of therapeutic agents to each aliquot, and determining whether the added agent(s) change a coagulation status of the plasma sample using waveform analysis. For example, a patient suffering from hemophilia can have his or her plasma tested by adding different amounts of recombinant factor VIII to the plasma and then performing the methods described herein.

In general, the therapeutic agent can be any agent used to treat a patient with a blood coagulation disorder. For example, the therapeutic agent can be an experimental agent.

The therapeutic agent can be a procoagulant, such as, for example, factor VIII, a variant thereof, factor IX, a variant thereof, factor FVII, a variant thereof, (such as activated factor VII variants), and/or a combination thereof. The protein factors can include recombinant or plasma-derived proteins. The therapeutic agent can be a mixture such as prothrombin complex concentrate (PCC), e.g. ProfilIX, or activated prothrombin complex concentrate (aPCC) or FVIII bypass agent (FEIBA) used to induce coagulation. The agents listed serve merely as examples, and are by no means exhaustive, and can include any agent promoting coagulation.

The therapeutic agent can be an engineered protein for treating a hemophilia A patient who has antibodies against FVIII, such as BAY 86-6150 available from Bayer HealthCare Pharrmaceuticals, Berlin, Germany.

Factor VIII (FVIII) variants, such as genetic variants, can be created by making a genetic variation of the recombinant FVIII gene constructs, resulting in, for example, B-domain deleted factor VIII and/or mutated factor VIII. The factor VIII variants can include, for example, variants of factor VIII modified post expression, such as, for example, FVIII with covalently attached polyethylene glycol (PEG), otherwise known as PEGylated PEG-conjugated factor VIII (FVIII), or PEG-FVIII. PEG-FVIII is a recombinant FVIII molecule that has prolonged circulatory life, e.g., is metabolized or eliminated more slowly from plasma, and is therefore, longer-acting compared to plasma FVIII. An example of such is BAY 94-9027 available from Bayer HealthCare Pharmaceuticals, Berlin, Germany. Factor VIII variant can also include fusion proteins with co-expressed binding elements.

The therapeutic agent can be an anti-coagulant, such as, for example, heparin, warfarin, rivaroxaban, and/or a combination thereof.

In certain embodiments, for example, such a method can correlate shifts in optical density waveforms (which are examples of coagulation status of a plasma sample) with increased procoagulant or anticoagulant activity by repeating the method with various amounts of procoagulants or anticoagulants. For example, that coagulation status can be compared to that of the patient's plasma, without the anticoagulant or procoagulant. In some instances, coagulation status of the test plasma can be compared to that of normal plasma.

In certain embodiments, a method is provided to monitor changes in an individual patient's coagulation status with procoagulant or anticoagulant therapy, such as patients with hemophilia A treated with a procoagulant (e.g., rFVIII), by obtaining plasma from the patient undergoing procoagulant or anticoagulant therapy and determining a coagulation status of that plasma using waveform analysis. That coagulation status can be compared to that patient's plasma's coagulation status before receiving the procoagulant or anticoagulant therapy or to the coagulation status of normal plasma.

In certain embodiments, a monitoring method is provided that can generate and store patient-specific coagulation signatures which can be compared over time or between different patients in a defined population (e.g., a hemophilia population). A coagulation status of a patient's plasma can be determined using waveform analysis. Additional coagulation status can be obtained over time on the same patient's plasma. The coagulation status of that patient can be compared to the coagulation status of plasma samples from patients with the same condition. The ability to track individual patient response to treatment over time can permit correlation of patient coagulation status with specific clinical symptoms. For example, for patients with a bleeding disorder, a specific clot signature can correlate with the onset of a bleeding episode, when the therapy provided is inadequate. A similar correlation can be employed for hypercoagulable patients undergoing some anticoagulant therapy.

### Drug Discovery Methods

In certain embodiments, a drug-discovery method is provided to screen for a therapeutic compound to treat a coagulation blood disorder. For example, one or more potential therapeutic compounds for treating a blood coagulation disorder can be added to the plasma sample of a patient prior to performing the coagulation assay, and waveform analysis can be used to determine whether the added compound changed a coagulation status of the plasma sample. The coagulation status obtained with specific therapeutic compounds present can be compared with those obtained from other therapeutic compounds or to the coagulation status of other plasma samples, including that of a normal control plasma sample. The therapeutic compound can be, for example, a mutant blood clotting factor, such as a mutant factor VIII protein.

In certain embodiments, a method is provided to screen for a dosage and/or efficacy of an anticoagulant. For example, an anticoagulant can be added, with or without adding a procoagulant, to a plasma sample (which can be normal plasma) prior to performing a coagulation assay such as dPT. Based on waveform analysis, a determination can be made as to whether the added procoagulant changed a coagulation status of the plasma sample. The coagulation status obtained with both procoagulant and anticoagulant added can be compared to a coagulation status obtained with no added factors, or with no added anticoagulant, for example. The added anticoagulant or procoagulant can be any anticoagulant or procoagulant, including those described herein, and/or a new compound being tested as a modulator of coagulation.

As stated, in some embodiments, the system 100 can include computer program code for performing all or a portion of the methods described herein. Such computer program code can reside in memory 106 in some embodiments. For example, the waveform analysis tool 104 can be configured to receive measured data from the coagulation analyzer 102 and to analyze the measured data to determine a coagulation status of a coagulation assay not provided by the coagulation analyzer 102. For example, in some embodiments the waveform analysis tool 104 can be is configured to (1) collect multiple sets of measured coagulation data for multiple plasma samples; (2) plot multiple sets of measured coagulation data for multiple plasma samples on one or more graphs; and/or (3) identify slope, minima, maxima and area under curve for measured coagulation data. Such data can be stored in one or more databases 108 in one or more embodiments. Using this and/or other data, the waveform analysis tool 104 can be configured to and/or otherwise assist a user to:
diagnose bleeding disorders based on measured coagulation data;
screen for bleeding disorders based on measured coagulation data;
discriminate between different coagulation factor deficiencies based on measured coagulation data;
discriminate between discrete levels of coagulation factors based on measured coagulation data;
diagnose treatment methods based on measured coagulation data;
discriminate between hemophiliac plasma, with and without inhibitors, and/or with or without therapeutic proteins used to treat hemophilia, based on measured coagulation data;
discriminate between different activators of coagulation based on measured coagulation data;
monitor effects of therapeutic agents based on measured coagulation data;
monitor tailored and/or patient specific therapies based on measured coagulation data;
monitoring therapeutic dosing based measured on coagulation data;
screen for new therapeutic compounds to treat coagulation blood disorders based on measured coagulation data;
screen for a dosage and/or efficacy of new anticoagulants or procoagulants based on measured coagulation data; and/or
screen for efficacy of new anticoagulants or procoagulants based on measured coagulation data.

All literature and similar materials cited in this application, including but not limited to, patents, patent applications, articles, books, treatises, and internet web pages, regardless of the format of such literature and similar materials, are expressly incorporated by reference in their entirety for any purpose.

The section headings used herein are for organizational purposes only and are not to be construed as limiting the subject matter described in any way.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

The invention claimed is:

1. A system for performing waveform analysis on coagulation data comprising:
a coagulation analyzer configured to measure at least one of turbidity and optical density of a coagulation assay and to output the measured data; and
a waveform analysis tool coupled to the coagulation analyzer and configured to receive the measured data, the waveform analysis tool configured to analyze the measured data to determine a coagulation status of the coagulation assay not provided by the coagulation analyzer.

2. The system of claim 1 wherein the waveform analysis tool is configured to perform at least one of:
collecting multiple sets of measured coagulation data for multiple plasma samples;
plotting multiple sets of measured coagulation data for multiple plasma samples on one or more graphs; and
identifying slope, minima, maxima and area under curve for measured coagulation data.

3. The system of claim 1 wherein the waveform analysis tool is configured to perform at least one of:
diagnosing bleeding disorders based on measured coagulation data; and
screening for bleeding disorders based on measured coagulation data.

4. The system of claim 1 wherein the waveform analysis tool is configured to perform at least one of:
discriminating between different coagulation factor deficiencies based on measured coagulation data; and
discriminating between discrete levels of coagulation factors based on measured coagulation data.

5. The system of claim 1 wherein the waveform analysis tool is configured to perform diagnosing of treatment methods based on measured coagulation data.

6. The system of claim 1 wherein the waveform analysis tool is configured to perform at least one of:
discriminating between hemophiliac plasma, with and without inhibitors, and with or without therapeutic proteins used to treat hemophilia, based on measured coagulation data; and
discriminating between different activators of coagulation based on measured coagulation data.

7. The system of claim 1 wherein the waveform analysis tool is configured to perform at least one of:
monitoring effects of therapeutic agents based on measured coagulation data;
monitoring tailored or patient specific therapies based on measured coagulation data; and
monitoring therapeutic dosing based measured on coagulation data.

8. The system of claim 1 wherein the waveform analysis tool is configured to perform at least one of:
screening for new therapeutic compounds to treat coagulation blood disorders based on measured coagulation data;
screening for a dosage and/or efficacy of new anticoagulants or procoagulants based on measured coagulation data; and
screening for efficacy of new anticoagulants or procoagulants based on measured coagulation data.

9. A method comprising:
obtaining a plasma sample from a patient;
performing a coagulation assay on the plasma sample;
measuring a coagulation property of the plasma sample using a coagulation analyzer so as to generate measured data;
performing waveform analysis on the measured data so as to obtain turbidity characteristics; and
employing the waveform analysis to determine a coagulation status of the coagulation assay not provided by the coagulation analyzer.

10. The method of claim 9, wherein the coagulation assay includes one or more of an activated partial thromboplastin time (“aPTT”) assay, a prothrombin time (“PT”) assay, a dilute prothrombin (“dPT”) assay, and a factor specific coagulation assay.

11. The method of claim 9, wherein the measured coagulation property includes turbidity.

12. The method of claim 9, wherein the measured coagulation property includes optical density.

13. The method of claim 9, wherein performing waveform analysis includes at least one of:
collecting multiple sets of measured coagulation data for multiple plasma samples;
plotting multiple sets of measured coagulation data for multiple plasma samples on one or more graphs; and
identifying slope, minima, maxima and area under curve for measured coagulation data.

14. The method of claim 9, wherein employing the waveform analysis to determine a coagulation status of the coagulation assay includes at least one of:
diagnosing bleeding disorders based on measured coagulation data; and
screening for bleeding disorders based on measured coagulation data.

15. The method of claim 9, wherein employing the waveform analysis to determine a coagulation status of the coagulation assay includes at least one of:
discriminating between different coagulation factor deficiencies based on measured coagulation data; and
discriminating between discrete levels of coagulation factors based on measured coagulation data,

16. The method of claim 9, wherein employing the waveform analysis to determine a coagulation status of the coagulation assay includes diagnosing treatment methods based on measured coagulation data.

17. The method of claim 9, wherein employing the waveform analysis to determine a coagulation status of the coagulation assay includes at least one of:
discriminating between hemophiliac plasma, with and without inhibitors, and with or without therapeutic proteins used to treat hemophilia, based on measured coagulation data; and
discriminating between different activators of coagulation based on measured coagulation data.

18. The method of claim 9, wherein employing the waveform analysis to determine a coagulation status of the coagulation assay includes at least one of:
monitoring effects of therapeutic agents based on measured coagulation data;
monitoring tailored or patient specific therapies based on measured coagulation data; and
monitoring therapeutic dosing based measured on coagulation data.

19. The method of claim 9, wherein employing the waveform analysis to determine a coagulation status of the coagulation assay includes at least one of:
screening for new therapeutic compounds to treat a coagulation blood disorder based on measured coagulation data;
screening for a dosage and/or efficacy of new anticoagulants or procoagulants based on measured coagulation data; and
screening for efficacy of new anticoagulants or procoagulants based on measured coagulation data.

20. The method of claim 9, wherein employing the waveform analysis to determine a coagulation status of the coagulation assay includes comparing the coagulation status of plasma samples from patients with the same condition.
